(19) United States
(12) Patent Application Publication
(10) Pub. No.: US 2023/0210832 A1
(43) Pub. Date: Jul. 6, 2023

(54) METHODS FOR TREATING ALZHEIMER'S DISEASE

(71) Applicants: Wilfred JEFFERIES, Surrey (CA); Chaahat SINGH, Surrey (CA)

(72) Inventors: Wilfred JEFFERIES, Surrey (CA); Chaahat SINGH, Surrey (CA)

(21) Appl. No.: 17/915,351

(22) PCT Filed: Mar. 29, 2021
(86) PCT No.: PCT/CA2021/050405

§ 371 Date: Sep. 28, 2022

Related U.S. Application Data
(60) Provisional application No. 63/002,449, filed on Mar. 31, 2020.

(51) Int. Cl. A61K 31/4439; A61K 31/404; A61K 45/06; A61P 25/28

(52) U.S. Cl. CPC A61K 31/4439; A61K 31/404; A61K 45/06; A61P 25/28

(57) ABSTRACT

The present invention provides a method of treating or delaying the onset of Alzheimer's disease or other neurological diseases in a patient having or at risk of developing Alzheimer's disease or other neurological diseases comprising administering to the subject an effective amount of an anti-angiogenic agent. In particular, the present invention provides a method of treating and/or delaying the onset of Alzheimer's disease or other neurological diseases by inhibiting Angiopoietin-2 mediated Tie-2 angiogenic pathway comprising administering to the subject an effective amount of an inhibitor of the Angiopoietin-2 mediated Tie-2 angiogenic pathway.

### FIELD OF THE INVENTION

The present invention relates to the field of therapeutics, in particular as they relate to Alzheimer's disease.

### BACKGROUND OF THE INVENTION

Alzheimer's disease (AD), a progressive neurodegenerative disorder of the elderly, causes loss of memory and other intellectual abilities leading to dementia. The exact cause of this disease is still unknown, and the mechanism of pathogenesis is highly debated. Amyloid beta (Aβ) peptide is central to the disease along with the cerebrovascular dysfunction and impaired cerebral blood flow (CBF). A strong link has been shown between brain vascular dysfunction and AD. This link has been established due to evidence of reduced blood-brain barrier (BBB) integrity preceding various AD neuropathologies. Furthermore, BBB dysfunction could influence CBF, which could in turn influence the blood vessel growth. Current dogma holds that AD BBB leakiness is likely due to vascular deterioration. Inflammatory changes in the AD brain lead to up-regulation of mediators, like vascular endothelial growth factor and tyrosine kinase with immunoglobulin-like and EGF-like domains-2 receptor (Tie-2), that initiate pathological angiogenesis. Studies indicate that pathological angiogenesis and BBB disruption occur as a compensatory response to impaired CBF. AD-induced neuroinflammatory responses promote the generation of reactive oxygen species and further endothelial damage. Aβ is shown to be a modulator of blood vessel density and vascular remodeling via angiogenic mechanisms. Studies on the cerebrovascular integrity of an AD mouse model showed a significant increase in the incidence of disrupted tight junctions. This disruption has been directly linked to neo-angiogenesis and an overall increase in microvascular density. This strongly supports amyloidgenesis-triggered angiogenesis as the basis of BBB disruption.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide methods of treating Alzheimer's disease.

In accordance with an aspect of the present invention, there is provided a method of treating and/or delaying the onset of Alzheimer's disease by inhibiting Angiopoietin-2 mediated Tie-2 angiogenic pathway comprising administering to the subject an effective amount of an inhibitor of the Angiopoietin-2 mediated Tie-2 angiogenic pathway.

In accordance with an aspect of the present invention, there is provided a method of treating and/or preventing cognitive decline by inhibiting cerebral neo-angiogenesis, comprising administering to the subject an effective amount of an inhibitor of the Angiopoietin-2 mediated Tie-2 angiogenic pathway, wherein cerebral neo-angiogenesis is inhibited by inhibiting the Angiopoietin-2 mediated Tie-2 angiogenic pathway.

In accordance with an aspect of the present invention, there is provided a diagnostic method for identifying subjects at risk of developing Alzheimer's disease or having early stage Alzheimer's disease or other neurological diseases, comprising screening for biomarkers from the Angiopoietin-2 mediated Tie-2 angiogenic pathway.

In accordance with an aspect of the present invention, there is provided a method of treating and/or preventing cognitive decline by inhibiting cerebral neo-angiogenesis with an inhibitor of VEGFR selected from the group consisting of Axitinib, Sunitinib and DC101.

In certain embodiments, the subject may be a human or animal. Optionally the animal is a companion animal such as a cat or dog.

### BRIEF DESCRIPTION OF THE DRAWINGS

These and other features of the invention will become more apparent in the following detailed description in which reference is made to the appended drawings.

FIG. 1: Pro-angiogenic effector expression increased in the AD mouse model, Tg2576. a, b) Level of phosphorylation of various kinases and expression levels of proteins involved in angiogenesis in 11-month-old untreated Tg2576 mice versus the untreated age-matched WT littermates, depicted as fold change. The data represent the mean fold change±S.E.M in the Tg2576 compared to the WT from three separate experiments. c. Immunoblots for a few effector molecules involved in modulating angiogenesis.

FIG. 2: Human brain endothelial cells treated with Aβ1-16 peptide (soluble amyloid species) upregulation of key angiogenic receptors, Tie-2 and VEGFR2, and increased expression of Ang-2 and neoangiogenic marker CD105. a. Western blotting analysis of proteins of interest in human brain endothelial cell (HBEC-5i) lysates from in vitro treatments is shown here. Histograms are quantitative representation of expression levels of the target proteins. b Western blotting analysis of Tie-2 and phosphorylated Tie-2 in lysates of HBEC-5i cells post treatment with Aβ1-42 and Aβ1-16 and Ang-2 FIG. 3. Brains of AD patients show co-localization of Tie-2 with Ang-2 and CD-105 along with increased vascularity and discontinuous tight junction. a) Immunostaining of Human cerebellum of normal and AD brains; Tie-2 (red), Ang-2 (blue) and CD-105 (green). The yellow arrows show the co-localization of Tie-2, Ang-2 and CD-105 in the AD brain panel. The white circles indicate no co-localization between CD105 and Tie-2 in the normal brain b, c) Micrographs show the human cerebellum of the normal and AD brains stained for CD-31 (green), Aβ (red) and occludin (blue). The normal brain (b) shows a continuous expression of occludin in an intact vessel as indicated by the circles whereas the AD brain (c) shows a discontinuous expression of occludin in an intact vessel, as indicated by the yellow arrows.

FIG. 4: Amyloid shows physical interaction with angiogenesis initiator receptor Tie-2.a) Western blot analyses were performed to examine the expression levels of Ang-1. b) Reciprocal pull-downs of Tie-2 and VEGFR2. Co-immunoprecipitation of Aβ with angiogenic receptors-Tie-2 and VEGFR2 in brain homogenates of 11-month-old vehicle-treated Tg2576 mice and age-matched vehicle-treated WT mice. c) The proximity ligation assay (PLA): Interaction between Aβ & Ang-2 (left panels) and Aβ & Tie-2 (right panels) in HBEC-5i cells that were treated with soluble AD. Fluorescence seen in red in the micrographs is indicative of the interactions between the two sets of proteins while blue is nuclear counterstaining with DAPI.

FIG. 5: Treatment with the anti-angiogenic drug, Axitinib, reduces cognitive impairment in aged Tg2576 mice. Pre-treatment 10-month old mice and Post-treatment 11-month old mice were assessed for their cognitive status, using tests for the analysis of different memory aspects. The data were pooled from 3 different trials and is represented as the mean±standard deviation. Statistical analysis was carried out using a 2-way ANOVA with correction for multiple comparisons using the Bonferroni's test (*p<0.03; **p<0.002; ***p<0.0002; ****p<0.0001).a) Open Field Test. WT mice (B6/SJL) spent less time in the center of the field, with no significant difference seen pre-treatment as well as when treated with Axitinib. Vehicle-treated Tg2576 mice spent significantly more time exploring the center than Axitinib-treated Tg2576 mice or WT mice. A significant difference was seen between the pre-treatment WT and Tg2576. A significant difference was also seen between pre-treatment Tg2576 and Axitinib-treated Tg2576 mice. There was no significant difference noted between the WT mice and Axitinib-treated Tg2576 mice. b) Representative track plots from the open field test. Cognitively aware WT and Axitinib-treated Tg2576 mice spend less time exploring the open center of the test arena and more time exploring the edges than cognitively-impaired pre-treatment and vehicle-treated Tg2576 mice. Each box represents the track from a different mouse. c) Spontaneous alternation (Y-maze) Test. A high percentage of alternation seen in the pre-treatment WT mice, with no significant difference observed with the vehicle and Axitinib treatment. Pre-treatment and vehicle-treated Tg2576 mice exhibited poor performance on the test, with a significantly lower percentage of alternation compared to the pre-treatment and vehicle-treated WT mice respectively. Axitinib-treated Tg2576 mice showed a significantly higher rate of alternation compared to the pre-treatment and vehicle-treated Tg2576 mice. No significant difference was observed between the WT mice and the Axitinib-treated Tg2576 mice. d) Contextual Fear conditioning Test. Pre-treatment, vehicle-treated and Axitinib-treated WT mice exhibited good associative memory by showing high freezing percentages. The pre-treatment and vehicle-treated Tg2576 mice displayed a significantly lower freezing percentage compared to the pre-treatment and vehicle-treated WT mice respectively. Axitinib-treated Tg2576 animals performed similarly to the WT mice. Axitinib-treated Tg2576 mice showed significantly higher performance compared to pre-treatment and vehicle-Tg2576 mice. e, f) Radial arm water maze. Radial arm water maze. (e) the time it takes for the mice to locate the escape platforms i.e. Latency time, and (f) the number of errors (working and reference memory errors) made by the mice when locating the escape platforms. Pre-treatment, vehicle- and Axitinib-treated WT mice showed a significant decrease in the latency time and a number of errors made when comparing test day 1 and test day 5. A similar improvement over the course of the trial was seen in the Axitinib-treated Tg2576 mice. No significant difference between test day 1 and test day 5 were seen in the pre-treatment and the vehicle-treated Tg2576 mice.

FIG. 6: Treatment of Tg2576 mice with the anti-angiogenic drug, Axitinib, reduces expression of AD, angiogenic marker, CD105 and tight junction proteins, ZO1 and occludin in aged Tg2576 mice. Brains from perfused mice were used for molecular analysis. Homogenates were used for western blotting analysis. The data are representative of means of individual animals with error bars representing the standard deviation from three separate experiments with WT n=6 and Tg2576 n=6 per treatment group. (*p<0.03; **p<0.002; ***p<0.0002; ****p<0.0001).a) Expression of the angiogenesis marker, CD105, was significantly higher in brains of vehicle-treated Tg2576 mice than in the brains of Axitinib-treated Tg2576 or WT animals. b) The expression of tight junction protein, ZO1, was significantly lower in vehicle-treated Tg2576 mice compared to the WT mice or Axitinib-treated Tg2576 mice. c) The presence of amyloid in the Tg2576 mouse brain was much higher in vehicle-treated than in Axitinib-treated animals, in which levels resembled those seen in WT brains. Representative blots are shown for mice from different groups.

FIG. 7: Immunofluorescence analysis shows Axitinib treatment reduces amyloid load, angiogenic cerebral vascularity and increased tight junction protein expression in aged Tg2576 mice. The micrographs here show the voxels of the field of view of the imaged sections represented by the grid. The data were pooled from 3 different trials and represented as the mean±standard deviation. Statistical analysis was carried out using unpaired Student's t test with WT n=6 and Tg2576 n=6 per treatment group. (*p<0.03; **p<0.002; ***p<0.0002; ****p<0.0001). a. Brain sections of mice from different groups were stained for the combination of markers CD105, Aβ and CD31. The micrograph panels are representative of the cortical and hippocampal regions of the brains from the mice belonging to the different treatment groups. Heavy amyloid Aβ staining and more Aβ plaques were seen in the vehicle-treated Tg2576 mouse brain compared to the brain of Axitinib-treated Tg2576 or WT mice. There was no difference in the overall expression of the mature vessel marker (CD31) between the groups, but the expression of the sprouting vessel marker (CD105) was much greater in vehicle-treated Tg2576 mice compared to Axitinib-treated Tg2576 or WT mice. The white grid is representative of the 3D volume of the field of view. Co-localization of CD105 (green), Aβ (red) and CD31 (blue) is indicated as bright purple. b. Brain sections of mice from different groups were stained for the combination of markers CD105, CD31 and the tight junction protein, occludin. The micrograph panels are representative of the cortical and hippocampal regions of the brains from the mice belonging to the different treatment groups. White arrows indicate a normal continuous occludin expression. Significantly lower occludin expression was noted in the vehicle-treated Tg2576, as compared to WT (B6/SJL) or Axitinib-treated Tg2576 mice. The micrographs are shown in 2D.

FIG. 8: Axitinib reduces the loss of tight junction structure and maintains the functionality of the BBB in aged Tg2576 mice. a. Brains from perfused mice were used for immunofluorescence analysis of the BBB. Brain sections were stained for the combination of markers CD105, CD31 and the tight junction protein, occludin. WT n=5 and Tg2576 n=6. The data are representative of three separate experiments (*p<0.03; **p<0.002; ***p<0.0002; ****p<0.0001). The first panel shows the merged micrograph with CD31 (red), CD105 (green) and Occludin (blue) staining and the other two panels are CD105 and Occludin where the field of view was magnified, as indicated by the superimposed grid, to have a more detailed view of the structure of a normal Occludin expression pattern observed in the WT mice (indicated by white arrows) and an abnormal Occludin expression as seen in the vehicle-treated Tg2576 mice. The percentage of tight junction protein (TJP) disruption was significantly higher in vehicle-treated Tg2576 mice compared to Axitinib-treated Tg2576 mice or WT mice. Note: the figure shows a magnified field of view of the micrograph in FIG. 3b. We are assessing the integrity of the tight junctions as opposed to the expression level of occludin protein in FIG. 3b. b) The absorbance of the dye was read with an ELISA plate reader (Spectra Max 190; Molecular Devices, Sunnyvale, Calif.) at 620 nm. The readings were divided by the weight of the brain. This experiment was repeated twice. (*p<0.03; **p<0.002; ***p<0.0002; ****p<0.0001). An increase in the uptake of Evans Blue in the CNS of the vehicle-treated Tg2576 mice was indicated by the high absorbance of the dye present in the homogenates. This implied that an increased BBB permeability or leakiness in vehicle-treated Tg2576 animals compared to the WT (B6/SJL) mice. No difference was seen between the vehicle- and drug-treated B6/SJL mice. Axitinib treatment of the Tg2576 mice resulted in less Evans Blue staining than seen in the vehicle-treated animals, thereby indicating a functioning BBB. c) The harvested brains from WT mice showed no colouration; however, vehicle-treated Tg2576 mice showed an overall bluish colouration of the brain. d) The micrographs here show the voxels of the field of view of the imaged sections represented by the grid. The micrographs are representative of brain sections from mice from a separate experiment, where both Tg2576 and WT were treated with Axitinib or vehicle, were stained for albumin in the CNS. Red indicates immuno-stained albumin that has leaked into the brain, and cyan indicates cell nucleus (DAPI). Axitinib treatment is associated with less albumin leakage into the brain, indicative of a more functional BBB, in contrast to the greater albumin staining in the vehicle-treated Tg2576

FIG. 9: Angiogenesis is positively correlated with amyloid beta and negatively correlated with tight junction proteins. Micrographs representing hippocampal and cortical co-localization of a) CD105 (green), Aβ (red) and CD31 (blue) b) CD105 (green), occludin (blue) and CD31 (red). The first column represents the micrographs showing the brain stained with a combination of antibody staining. The white overlay indicates the area in the field of view that was magnified and shown in the next two columns labeled either CD105 and Aβ or CD105 and Occludin. Comparing the brain sections from 11-month-old vehicle-treated Tg2576 mice and vehicle-treated WT littermates using total fluorescence volume, the Thresholded Pearson's Correlation Coefficient was calculated to assess the correlation between Aβ and neo-angiogenic marker, CD105 and between neo-angiogenic marker, CD105 and tight junction protein, Occludin. This indicated that in Tg2576 mice, a positive correlation coefficient of r=+0.62 was seen between Aβ and CD105 and a negative correlation coefficient of r=−0.73 was seen between CD105 and occludin.

FIG. 10: Schematic depicting a model mechanism for amyloid beta causing pathological angiogenesis and disruption of the tight junction proteins and the breakdown of the neurovascular unit. a) Normal physiological activation of blood vessel growth by Angiopoietin-1 activating Tie-2. b) The initiation of pathological and destabilized blood vessel growth. Increased levels of amyloid beta seen interacting with cerebral vessels, in the presence of hypoxic conditions and low levels of Angiopoietin-1 recruits VEGF in the vicinity of the Tie-2 receptor. This assists Angiopoietin-2 to bind to Tie-2 and activate destabilized vessel formation, which is indicated by the increase in the downstream signaling pathways and activation of pro-angiogenic transcription factors for endothelial survival, proliferation, migration and production of VEGF. VEGF effector moves out into the extracellular space, further facilitating Ang-2 in continuously binding to Tie-2 receptor molecules and constitutively activating endothelial cells, leading to the formation of pathogenically sprouting vessels. Increases in certain effector molecules like FAK lead to rearrangement of the actin cytoskeleton that maintains the physical intactness of the tight junctions and thus destabilizes the vessels, thereby increasing vascular permeability. This allows for the toxic blood molecules to enter into the central nervous system and cause inflammation, hypoxia and neuronal pathologies. In this way, angiogenesis culminates in AD pathology and cognitive decline.

FIG. 11: Treatment with the anti-angiogenic drug, Sunitinib, reduces cognitive impairment in aged Tg2576 mice. 10-month-old Tg2576 and WT littermate mice were treated with the anti-angiogenic tyrosine kinase inhibitor, Sunitinib, for 1 month at a dose of 80 mg/kg, 3 days/week. Pre-treatment 10-month old mice and Post-treatment 11-month old mice were assessed for their cognitive status, using tests for the analysis of different memory aspects. The data were pooled from 3 different trials and is represented as mean±standard deviation. Statistical analysis was carried out using a 2-way ANOVA with correction for multiple comparisons using the Bonferroni's test (*p<0.03; **p<0.002; ***p<0.0002; ****p<0.0001). For open field test, fear conditioning and RAWM tests: pre-treatment-WT, n=8; WT-vehicle, n=6; WT-Sunitinib, n=7; pre-treatment-Tg2576, n=8; Tg2576-vehicle, n=9; Tg2576-Sunitinib, n=11. For Y-maze: pre-treatment-WT, n=8; WT-vehicle, n=11; WT-Sunitinib, n=10; pre-treatment-Tg2576, n=8; Tg2576-vehicle, n=11; Tg2576-Sunitinib, n=15). a) Open Field Test. WT mice (B6/SJL) spent less in the center of the field, with no significant difference seen pre-treatment as well as when treated with Sunitinib. Vehicle-treated Tg2576 mice spent more time exploring the center but this was not seen when the Tg2576 mice were treated with Sunitinib. A significant difference was seen between the pre-treatment WT and Tg2576. A significant difference was also seen between pre-treatment Tg2576 and Sunitinib-treated Tg2576 mice. There was no significant difference noted between the Sunitinib-treated WT and Sunitinib-treated Tg2576 mice. However, a significant difference was observed between vehicle-treated and Sunitinib-treated Tg2576 mice. Similar results were observed in the number of entries in the central squares and the total distance travelled by the different group of mice. b) Spontaneous alternation (Y-maze). Cognitively aware mice show a high percentage of alternation, as was seen in the pre-treatment, vehicle-treated and Sunitinib-treated WT mice. There was a significant difference between the pre-treatment WT and Tg2576 mice as well as between the vehicle-treated WT and Tg2576 mice. A significant increase in the percentage alternation was seen in the Sunitinib-treated Tg2576 mice compared to the vehicle-treated Tg2576 mice, however, there was no significant difference seen between the Sunitinib-treated and pre-treatment Tg2576 mice. Sunitinib-treated Tg2576 mice did not reach the level of performance as the Sunitinib-treated WT mice as a significant difference was seen between the two groups. c) Contextual Fear conditioning. WT mice, both pre-treatment and vehicle-treated, showed high freezing percentages indicative of good associative memory. Sunitinib-treated WT mice showed a significant increase in freezing percentage compared to the vehicle-treated WT mice but not when compared to pre-treatment. Pre-treatment and vehicle-treated Tg2576 mice displayed a significantly lower freezing percentage compared to pre-treatment and vehicle-treated WT mice respectively. A significant increase was seen in the Sunitinib-treated Tg2576 mice when compared to vehicle-treated Tg2576 mice, however, this increase was not significant when compared to pre-treatment Tg2576 mice. A significant difference still remained between the Sunitinib-treated WT and the Sunitinib-treated Tg2576 mice. d, e) Radial arm water maze. (d) the time it takes for the mice to locate the escape platforms i.e. Latency time, and (e) the number of errors (working and reference memory errors) made by the mice when locating the escape platforms. Pre-treatment, vehicle- and Sunitinib-treated WT mice showed a significant decrease in the latency time and the number of errors made when comparing test day 1 and test day 5. A similar improvement over the course of the trial was seen in the Sunitinib-treated Tg2576 mice. No significant difference between test day 1 and test day 5 were seen in the pre-treatment and the vehicle-treated Tg2576 mice. A significant difference was seen in the latency time and number of errors made on test day 5 between the pre-treatment WT and Tg2576 mice as well as the vehicle-treated WT and Tg2576 mice. A significant difference was also observed between the pre-treatment and Sunitinib-treated Tg2576 mice as well as between the vehicle-treated and Sunitinib-treated Tg2576 mice. No significant difference was observed when comparing latency time and number of errors made by the pre-treatment and vehicle-treated Tg2576 mice as well as the Sunitinib-treated WT and Tg2576 mice on test day 5

FIG. 12: Treatment with Sunitinib reduces expression of Aβ, angiogenic marker, CD105 and tight junction proteins, ZO1 and occludin in aged Tg2576 mice. Brains from perfused mice were used for molecular analysis. Homogenates were used for western blotting and fixed brain sections for histological analysis. Representative data from three separate experiments are shown for mice from the different groups with WT n=6 and Tg2576 n=6. The histograms show mean±standard deviation. Statistical analysis is done using unpaired Student's t test (*p<0.03; **p<0.002; ***p<0.0002; ****p<0.0001). Semiquantitative western blot analysis (a-c): a) Expression of CD105 and ZO1. Brain expression of CD105, an angiogenesis marker, was significantly lowered after Sunitinib treatment of Tg2576 mice, resembling the WT brain levels. The expression of tight junction protein, ZO1, was significantly lower in vehicle-treated Tg2576 mice compared to the WT mice and Tg2576 mice treated with Sunitinib. b) The presence of Aβ and c) the presence of APP was significantly less after Sunitinib treatment in comparison to vehicle-treatment in the Tg2576 mouse brain so that levels in the brains of Sunitinib-treated animals resembled those in WT animals. Immunofluorescence analysis (d): brain sections of mice from different groups were stained for the combination of markers CD105, Aβ and tight junction protein occludin. The micrograph panels shown are representative of the cortical and hippocampal regions of the brains from mice belonging to the different treatment groups. Heavy amyloid staining in the Tg2576 mouse brain compared to the WT mice. More sprouting vessels (CD105) and Aβ plaques and less occludin expression were seen in vehicle-treated Tg2576 compared to the WTs or Sunitinib-treated Tg2576 mice.

FIG. 13: Treatment of aged Tg2576 mice with the anti-angiogenic drug, Sunitinib, reduces the loss of tight junctions in cerebral vessels. The brain sections of mice from different groups were stained for the tight junction protein, occludin. The micrograph panels shown are representative of the cortical and hippocampal regions of the brains from mice belonging to the different treatment groups with WT n=6 and Tg2576 n=6. Disrupted occludin expression was noted in the vehicle-treated in Tg2576 as compared to WT mice (B6/SJL). In contrast, occludin expression in the Sunitinib-treated Tg2576 mice was similar to WT. Normal expression patterns of occludin are indicated with the white arrows in the micrographs.

FIG. 14: Functional BBB in aged Tg2576 mice after Sunitinib treatment a) Evans Blue dye was i.p. injected into mice from the treatment trial. Brains were harvested and homogenized with 50% trichloroacetic acid followed by dilution with ethanol. The absorbance was read with an ELISA plate reader (Spectra Max 190; Molecular Devices, Sunnyvale, Calif.) at 620 nm. The readings were divided by the weight of the brain. This experiment was repeated twice. Data represents mean±standard deviation. Statistical analysis was done using unpaired Student's t test (*p<0.03; **p<0.002; ***p<0.0002; ****p<0.0001). The absorbance of the dye in different groups. Greater uptake of Evans Blue in the CNS was seen in the brain homogenates of the vehicle-treated Tg2576 mice, higher BBB permeability or leakiness compared to the WT mice (B6/SJL) or Sunitinib-treated Tg2576 animals. b) Representative 2D micrographs of coronal brain sections from WT and Tg2576 mice treated with vehicle or Sunitinib, where green indicates albumin that has leaked into the brain and red indicates Evans blue dye. Sunitinib treatment of aged Tg2576 resulted in less albumin and Evans blue in the brain, indicative of a functional BBB, in comparison to vehicle-treated Tg2576 mice.

FIG. 15: Anti-angiogenic drug, DC-101, partially prevents the cognitive decline associated with aged AD mouse model Tg2576. 10-month-old Tg2576 and WT littermate mice were treated with DC-101, a monoclonal antibody that targets VEGFR-2, for 1 month at a dose of 0.8 mg/mouse, 3 days/week. After treatment, the mice were assessed for their cognitive status, using tests for the analysis of different memory aspects (*p<0.03; **p<0.002; ***p<0.0002; ****p<0.0001). The above data represents the mean±standard deviation from 3 different trials. Statistical analysis was done using 2-wat ANOVA (Pre-treatment WT and Tg2576, n=8; WT-vehicle and WT-DC101, n=10; Tg2576-vehicle and g2576-DC101, n=14). a) Open Field Test: Pre-treatment and vehicle-treated WT mice (B6/SJL) spend less in the center of the field, with no significant difference seen when treated with DC-101. Pre-treatment and vehicle-treated Tg2576 mice spend significantly more time exploring the center compared to the WT mice and DC-101-treated Tg2576 mice. A similar result was seen in the total distance travelled by the mice and the number of entries made in the center of the field. b) Spontaneous alternation (Y-maze): Cognitively aware mice show a high percentage of alternation, as was seen in the pre-treatment and vehicle-treated WT mice with no significant change observed after treatment with DC-101. Pre-treatment and vehicle-treated Tg2576 mice exhibited poor performance on the test, with a significantly lower percentage of alternation compared to the WT mice. DC-101-treated Tg2576 mice showed a significant difference compared to the vehicle-treated Tg2576 mice but no significant difference compared to the pre-treatment Tg2576 mice. c) Contextual Fear conditioning: WT mice, pre-treatment, vehicle-treated and DC-101-treated, showed high freezing percentages indicative of good associative memory. Pre-treatment and vehicle-treated Tg2576 mice displayed a significantly lower freezing percentage. DC-101-treated Tg2576 animals showed a significant difference compared to vehicle-treated Tg2576 mice but no significant difference compared to the pre-treatment Tg2576 mice.

FIG. 16: DC101 treatment of aged Tg2576 mice shows lower expression of Aβ, angiogenic vessel marker, CD105 and higher expression of tight junction proteins: Brains from perfused mice were used for molecular analysis. Homogenates were used for western blotting analysis and fixed brain sections were used for immunofluorescence analysis. The graphs show data as the mean±standard deviation and are pooled from three separate experiments with total WT n=12 and Tg2576 n=12. Statistical analysis was done using unpaired students t test. Semiquantitative western blot analysis (a-b): a) The presence of Aβ and CD105 in the Tg2576 mouse brain was significantly lowered by DC-101 treatment so that levels in the brain resembled the WT levels b) The expression of tight junction protein, ZO1, was significantly lower in vehicle-treated Tg2576 mice than DC-101-treated Tg2576 mice or WT mice. Immunofluorescence analysis (c): brain sections of mice from different groups stained for the combination of markers; CD105, Aβ and tight junction protein occludin. The micrograph panels shown are representative of the cortical and hippocampal regions of the brains from the mice belonging to the different treatment groups. Heavy amyloid staining and more Aβ plaques are seen in the vehicle-treated Tg2576 mouse brain compared to DC-101-treated Tg2576 or WT mice. More sprouting vessels (CD105) are seen in vehicle-treated Tg2576 compared to DC-101-treated Tg2576 or WTs. Substantially diminished occludin expression was noted in the vehicle-treated Tg2576 compared to WT mice (B6/SJL).

FIG. 17: Treatment of aged Tg2576 mice with the anti-angiogenic antibody, DC-101 reduces the loss of tight junctions in cerebral vessels. The brain sections of mice from different groups were stained for the tight junction protein, occludin. The micrograph panels shown are representative, of the cortical and hippocampal regions of the brains from the mice belonging to the different treatment groups with WT n=5 and Tg2576 n=6. Abnormal occludin expression was noted in the brains of vehicle-treated Tg2576 mice as compared to the WT (B6/SJL) or DC-101-treated Tg2576 mice. Normal expression pattern of occludin is indicated with the white arrows in the micrographs.

FIG. 18: Functional BBB in aged Tg2576 mice after DC-101 treatment Representative micrographs of coronal brain sections from mice (WT or Tg2576) treated with vehicle or DC-101, where green indicates immunostaining of albumin that has leaked into the brain and red indicates the Evans blue dye. Vehicle-treated and DC101-treated WT mice and DC101-treated Tg2576 mice show lower Evans blue dye and immune-stained albumin compared to vehicle-treated Tg2576 mice.

FIG. 19: Concentration of Axitinib quantitated in mouse plasma and brain. Pharmacokinetics analysis of Axitinib at 10 mg/kg/mouse at different time points to demonstrate the effectiveness and bioavailability of the chosen drug dosage. Concentration of Axitinib in the samples were calculated from the linear-regression calibration curve with internal standard calibration. AUC=area under the curve. The curve represents the metabolism of the drug over time and the AUC represents the total drug exposure over time in the system. The no-observed-adverse-effect-levels (NOAELs) for Axitinib in mice are set at <10 mg/kg/day for 26 weeks of once daily oral gavage administration.

FIG. 20: Angiogenesis is positively correlated with amyloid beta and negatively correlated with tight junction proteins in the Tg2576 mouse brain cortex.

a) Micrographs representing cortical region of Tg2576 mouse brains depicting co-localization of CD105 and AD. CD105 (green), Aβ (red) and merged where Red+Green=Yellow. Size of each micrograph: 100 μm*100 μm.

b) The scatterplot represents the number and intensity of pixels that are plotted the merged figure (a) of Aβ (red) versus CD105 (green). The Thresholded Pearson's Correlation coefficients are calculated from the scatterplots of 12 separate comparisons of Aβ versus CD105 and shown in the relative frequency histogram, for an average of (r)=+0.633 with a standard deviation of 0.096, p=0.01 (statistically significant, one-tail test calculated using free statistics calculator (Available from: Available from https://www.danielsoper.com/statcalc]).

c) Micrographs representing cortical region of Tg2576 mouse brains depicting co-localization of CD31 (red), CD105 (green), and Occludin (blue). Merged micrographs show Green+Red=Yellow; Red+Blue=Purple; Blue+Green=Cyan; and Red+Blue+Green=White. Size of each micrograph: 100 μm*100 μm.

d) The scatterplot represents the number and intensity of pixels that are plotted the merged figure for CD31 (red) vs Occludin (blue). The Thresholded Pearson's Correlation coefficients are calculated from the scatterplots of 13 separate comparisons of CD31 versus Occludin and shown in the relative frequency histogram, for an average of (r)=+0.552 with a standard deviation of 0.095, p=0.03 (statistically significant, one-tail test; calculated using free statistics calculator available from: Available from https://www.danielsoper.com/statcalc)

e) The scatterplot represents the number and intensity of pixels that are plotted the merged figure for CD105 (green) vs Occludin (blue). The Thresholded Pearson's Correlation coefficients are calculated from the scatterplots of 10 separate comparisons of CD105 versus Occludin and shown in the relative frequency histogram, for an average of (r)=−0.140 with a standard deviation of 0.073, p=0.34 (one-tail test not statistically significant.

### DETAILED DESCRIPTION OF THE INVENTION

Vascular dysfunction now appears to be a crucial pathological hallmark of a number of neurological diseases including but not limited to Alzheimer's Disease, and two key precursors to neurodegenerative changes and Aβ deposition in AD are BBB breakdown and CBF impairment. The present invention relates to the finding, described herein, that the Angiopoietin-2 mediated Tie-2 angiogenic pathway is aberrantly stimulated by an overproduction of Aβ in Alzheimer's Disease (AD) which leads to destabilized cerebral vessel formation and disrupted blood-brain barrier. It is further demonstrated herein that brain health and wellness can be improved by targeting this pathway enabling the reduction of Aβ deposits and reversing cognitive decline.

In certain embodiments, there is provided a method of treating and/or delaying the onset of Alzheimer's disease by inhibiting angiogenesis in the brain. In specific embodiments, there is provided a method of treating and/or delaying the onset of Alzheimer's disease by inhibiting the Angiopoietin-2 mediated Tie-2 angiogenic pathway. In certain embodiments, there is provided a method of treating and/or preventing cognitive decline by inhibiting cerebral neo-angiogenesis. In specific embodiments, cognitive decline relates to one or more of the following: spatial awareness, exploration, associative memory, working memory and reference memory. In specific embodiments, cognitive decline relates to one or more of the following: spatial awareness, exploration, associative memory, working memory and reference memory.

In specific embodiments, cerebral neo-angiogenesis is inhibited by inhibiting the Angiopoietin-2 mediated Tie-2 angiogenic pathway. This pathway may be inhibited by inhibiting the Angiopoietin-2 Tie-2 binding, blocking Tie2 signalling, blocking effector molecules, blocking soluble soluble Aβ interacting with Tie-2. In certain embodiments, the agents include, but are not limited to, antibodies that bind ANG-2 and/or TIE2 and fragments and variants thereof; non-antibody peptides that bind ANG-2, including, but not limited to, soluble TIE2 receptor and fragments and variants thereof, including the TIE2 extracellular domain; non-antibody peptides that bind TIE2, including ANG-2-based peptides; nucleotide-based inhibitors of ANG-2 expression; and small molecule inhibitors of ANG-2 or TIE2. In certain embodiments the inhibitors are siRNAs or microRNAs.

Exemplary agents that block the angiopoietin-2 tie2 pathway are known in the art and include but are not limited to Trebananib, Vanucizumab, MEDI3617, Nesvacumab, Rebastinib, MGCD-265; Pexmetinib; CEP-11981, BAY-826, 3,21-dioxo-olean-18-en-oic acid, Altiratinib, 4-[4-(6-methoxy-2-naphthalenyl)-2-[4-(methylsulfinyl)phenyl]-1H-imidazol-5-yl]-pyridine, AB536, 2×CON and L1-7

Trebananib is a peptide-Fc fusion protein that targets the Ang1/Ang2/Tie2 pathway and inhibits angiogenesis by blocking the interaction between Ang1/Ang2 with the Tie2 receptor. Vanucizumab is a bi-specific monoclonal antibody which binds Angiopoietin-2 (Ang2) and VEGF-A). MEDI3617 is a selective anti-angiopoietin-2 (Ang2) monoclonal antibody. Nesvacumab is a monoclonal antibody which targets angiopoietin 2. BAY-826 (described in WO201310590) is a TIE-2 inhibitor. CEP-11981 is a Pan-VEGFR/Tie2 tyrosine kinase inhibitor. Rebastinib is an inhibitor of Tie2 tyrosine kinase receptor. MGCD-265 is a multi-targeted tyrosine kinase inhibitor, inhibiting MET, VGFR1-3, Tie, and Ron. Altiratinib is an orally bioavailable inhibitor of c-Met/hepatocyte growth factor receptor (HGFR), vascular endothelial growth factor receptor type 2 (VEGFR2), Tie2 receptor tyrosine kinase (TIE2), and tropomyosin receptor kinase (Trk), with potential antiangiogenic and antineoplastic activities. Pexmetinib is an orally bioavailable small-molecule inhibitor of p38 and Tie2 kinases with potential antineoplastic, anti-inflammatory and antiangiogenic activities. 3,21-dioxo-olean-18-en-oic acid is a naturally occurring non-protein inhibitor of Tie2 kinase isolated from Acacia aulacocarpa. 4-[4-(6-methoxy-2-naphthalenyl)-2-[4-(methylsulfinyl)phenyl]-1H-imidazol-5-yl]-pyridine is a selective Tie2 tyrosine kinase inhibitor. are ANG-2 inhibitors Oliner et al. Cancer Cell 6(5):507-516).

In certain embodiments, the modulators of the Angiopoietin-2 mediated Tie-2 angiogenic pathway are natural products or extracts. Exemplary products or extracts include for example products or extracts from Acacia aulacocarpa, Artemisia annua (Chinese wormwood), Viscum album (European mistletoe), Curcuma longa (curcumin), Scutellaria baicalensis (Chinese skullcap), resveratrol and proanthocyanidin (grape seed extract), Magnolia officinalis (Chinese magnolia tree), Camellia sinensis (green tea), Ginkgo biloba, quercetin, Poria cocos, Zingiber officinalis (ginger), Panax ginseng, Rabdosia rubescens hora (Rabdosia), Chinese destagnation herbs, licorice (Glycyrrhiza, Leguminosae), Forsythia suspensa, Forsythia fructus, Voacanga africana. In certain embodiments the natural product is selected from voacangine, tannic acid, oleanolic acids, terpenes, saponins and glycyrrhizin.

In certain embodiments a VEGFR inhibitor is used alone or in combination to inhibit angiogenesis. Exemplary VEGFR inhibitors include but are not limited to a tyrosine kinase inhibitor such as Axitinib and Sunitinib and anti-VEGFR antibodies inhibit VEGFR such as the anti-VEGFR2 antibody DC101.

In certain embodiments, there is provided a method of treating and/or preventing cognitive decline by inhibiting cerebral neo-angiogenesis by inhibiting the Angiopoietin-2 mediated Tie-2 angiogenic pathway. In certain embodiments, there is provided a method of treating and/or preventing cognitive decline by inhibiting cerebral neo-angiogenesis with an inhibitor of VEGFR. Such inhibitors include but are not limited to Axitinib, Sunitinib and DC101.

In certain embodiments, there is provided a method of reducing cerebral vascular pathology, cerebral Aβ load and tight junction disruption in Alzheimer's Disease by inhibiting the Angiopoietin-2 mediated Tie-2 angiogenic pathway. In specific embodiments, provided a method of reducing cerebral vascular pathology, cerebral Aβ load and tight junction disruption in Alzheimer's Disease by a VEGFR inhibitor, including but not limited to Axitinib or Sunitinib.

The use of combinations of agents is also contemplated in certain embodiments of the invention. The agents may be administered separately or as a single composition.

The agents may be formulated as known in the art. Such formulations include but are not limited to drug delivery systems and cationic surfactants.

In certain embodiments there is provided biomarkers related to disease's associated with Angiopoietin-2 mediated Tie-2 angiogenic pathway, including but not limited to Alzheimer's disease and the diseases listed above. In an animal model of AD, the downstream signalling pathways MAPK, AKT, JAK/STAT and Wnt involved in cell proliferation, endothelial migration and cell survival were highly activated, as were transcription factors responsible for promoting vessel formation like STAT3, c-fos, CREB and p53. It was observed that there was upregulation of CD105, vascular endothelial growth factor (VEGF), fibroblast growth factor (FGF), Osteopontin, proliferation, platelet factor 4, angiogenin and IL-10, among other proangiogenic factors. The Tie-2 receptor ligand, Ang-1, was reduced in the animal model. There were also increases in APP, pERK-1, NOTCH-1 and p-FAK.

Accordingly, the biomarkers may include but not limited to proangiogenic factors and components of the Angiopoietin-2 mediated Tie-2 angiogenic pathway. In certain embodiments, the biomarkers include CD105, vascular endothelial growth factor (VEGF), fibroblast growth factor (FGF), Osteopontin, proliferation, platelet factor 4, angiogenin and IL-10, among other proangiogenic factors, Tie-2 receptor ligand, Ang-1, APP, pERK-1, NOTCH-1 and p-FAK.

In some embodiments, the invention provides for methods of identifying agents for the prevention and/or treatment of disease's associated with Angiopoietin-2 mediated Tie-2 angiogenic pathway that comprise testing the ability of a candidate agent to inhibit angiogenesis in the brain. In some embodiments, the invention provides for methods of identifying agents for the prevention and/or treatment of disease's associated with Angiopoietin-2 mediated Tie-2 angiogenic pathway that comprise testing the impact of the agent on selected biomarkers.

In some embodiments, the invention provides for methods of identifying agents for the prevention and/or treatment of Alzheimer's disease that comprise testing the ability of a candidate agent to inhibit angiogenesis in the brain. In some embodiments, the invention provides for methods of identifying agents for the prevention and/or treatment of Alzheimer's disease that comprise testing the impact of the agent on selected biomarkers.

Certain embodiments of the invention provide for diagnostic methods for identifying subjects at risk of developing Alzheimer's disease or having early stage Alzheimer's disease comprising screening for selected biomarkers.

Appropriate methods of screening biomarkers are known in the art and include but are not limited to a phospho-kinase and angiogenesis proteome array analysis.

### EXAMPLES

### Exemplary Methods

Mice, cells and human brain samples: The Tg2576 AD model mouse expresses the Swedish mutant of the amyloid precursor protein (K670N/M671L) (Hsiao et al., 1996; Hsiao et al., 1995) under control of the hamster prion protein promoter (Taconic). Mice were maintained on mixed C57B16/SJL background by mating heterozygous Tg2576 males to C57B16/SJL F1 females. Wild-type littermates were used as controls.

The genotyping protocol was performed as described by (Hsiao et al., 1995) by PCR: Briefly, two parallel PCR reactions were performed to distinguish heterozygote from wild-type. The PrP-APP fusion DNA (corresponding to the heterozygote) was amplified using primers 1502 (hamster PrP promoter, 5′-GTGGATAACCCCTCCCCCAGCCTAGACCA-3′) and 1503 (human APP, 5′-CTGACCACTCGACCAGGTTCTGGGT-3′). The primer combination 1502 and 1501 (mouse PrP, 5′-AAGCGGCCAAAGCCTGGAGGGTGGAACA-3′) was used as a positive control for the reaction.

Aged Tg2576 and wild-type mice of both sexes were used at 10 months of age.

Mouse numbers used in the respective Alzheimer's mouse experiments are noted in the figure legends. Mice were fed standard lab chow and water ad libitum and kept under a 12-hour light/dark cycle. All protocols and procedures involving the care and use of animals in these studies were reviewed and approved by the UBC Animal Care Committee.

Human brain endothelial cells, HBEC-5i (ATCC® CRL-3245™) were cultured on 0.1% gelatin coated tissue culture plates in DMEM-F12 media (Gibco; cat. #10565-018) supplemented by 10% heat-inactivated fetal bovine serum (FBS) (Gibco; cat. #A31607-01) and 40 μg/ml endothelial growth supplement (Sigma-Aldrich; cat. #E2759). Cells were grown until they reached 70% confluency and then used for experiments.

Human brain samples were obtained from BioChain® (Newark, Calif.) in the form of paraffin tissue section panels (cat #T8236446A1z) with Alzheimer's disease brain tissues and normal brain tissues.

Antibodies, peptides and chemicals: Antibodies used for western blots, immunofluorescence and immunoprecipitation (concentrations used as per the company datasheets unless indicated otherwise): anti-CD105/Endoglin (R&D systems; AF1097), anti-amyloid beta 6 E-10 (BioLegend; 39320), anti-ZO1 (ThermoFisher Scientific; cat. #61-7300), anti GAPDH antibody (abcam; ab181602), anti-actin (Santa Cruz; sc1615), anti-occludin (abcam; ab31721), anti-CD31 (abcam; ab28364), anti-albumin (abcam; ab19194), anti-phosphoTie2 (R&D systems; AF 3909), anti-Tie-2 (A&D systems; AF313; 1:100) anti-Tie2/TEK (Millipore; cat. #05-584), anti-amyloid (1-16) 6 E10 (Biolegend; cat. #803002), anti-amyloid (abcam; ab39377; 1:700), anti-VEGFR2 (abcam; ab11939), anti-pVEGFR2-phospho Y1214 (abcam; ab131241) anti-FAK (abcam; ab40794), anti-phospho FAK (abcam; ab81298), anti-VEGF-A165 (abcam; ab69479), anti-Ang-1 (abcam; ab8451), anti-Ang-2 (abcam; ab8452).

Peptides used for treating of HBEC-5i cells and used as positive controls for immunoblotting and immunoprecipitation: Aβ1-42 peptide (NovoPrep synthetic peptide; cat. #A-42-T-1), Aβ1-16 peptide (AnaSpec; AS-24225), human Ang-2 peptide (abcam; ab99482), human VEGFA peptide (abcam; ab46160).

Antibodies for FACS: FITC anti-mouse CD45.2 Antibody (Biolegend; cat. #109805), PE/Cy7 anti-mouse CD45.1 Antibody (Biolegend; cat. #110729).

Chemicals used: NP-40 (abcam; ab142227), 100× Halt protease and phosphatase inhibitor cocktail (ThermoFisher Scientific; cat. #78440).

Phospho-kinase and Angiogenesis Proteome Arrays: Total brain tissue homogenates were prepared as described above. Homogenates were incubated on nitrocellulose membranes containing different antibodies printed in duplicates provided with Proteome Profiler Human Phospho-Kinase Array Kit (R&D Systems; ARY003B) and Proteome Profiler Mouse Angiogenesis Array Kit (R&D Systems; ARY015). The Proteome Profiler Human Phospho-Kinase Array Kit (R&D Systems; ARY003B) can also be used for mouse samples. The phospho-kinase analysis and angiogenesis analysis were performed according to the manufacturer's instructions. Phospho-kinase levels and levels of proteins involved in angiogenesis in Tg2576 mice versus the WT littermates were depicted as fold change.

Cell culture: Human brain endothelial cells were grown in 10 cm tissue culture plates. The cells were treated with either PBS, Aβ1-42 peptide (10 μM), Aβ1-16 peptide (10 μM), Ang-2 peptide (200 ng/ml) or VEGFA-165 (100 ng/ml) peptide for 24 hrs in a starving medium. After the treatment, the medium was removed, and cells were washed with PBS and then lysed with RIPA buffer that contained protease and phosphatase inhibitors. These cells were then used to analyze different proteins of interest.

Immunoblotting analysis and co-immunoprecipitation: For western blot analysis, the fractional homogenate samples were prepared with Dithiothreitol (DTT) in the sample buffer (100 mM Tris-Cl (pH 6.8), 4% (w/v) SDS, 0.2% (w/v) bromophenol blue, 20% (v/v) glycerol and 200 mM DTT). Homogenates were electrophoresed and immunoblotted with indicated antibodies. For co-immunoprecipitation, homogenates were first pre-cleaned by incubating with a bead slurry and centrifuged at 14,000×g for 4 minutes at 4° C. Beads were discarded, and supernatant kept for co-immunoprecipitation. Protein A or protein G beads were washed twice with wash buffer (10 mM Tris HCl pH 7.4, 150 mM NaCl, 1% Nonidet P-40, 1 mM EDTA, 1× Halt protease and phosphatase inhibitor cocktail and 20 mM N-Ethylmaleimide), centrifuged at 3,000×g for 2 minutes at 4° C. and incubated with the indicated antibody for 4 hours at 4° C. on a rotating shaker. Beads were then washed twice and incubated with pre-cleaned homogenates overnight at 4° C. Beads were washed, and the complex was eluted by using 2×50 μl SDS loading buffer without DTT and heating at 50° C. for 10 minutes. A second elution was carried out by 2×SDS buffer in the presence of DTT. The proteins in the eluate were resolved on the acrylamide gel according to standard practices. Immunoblotting was performed on nitrocellulose membranes using primary antibodies against the various proteins. The signal intensities were imaged by using the Odyssey infrared image system (LICOR), and relative levels of immunoreactivity were analyzed by the Image Studio Lite software.

Pharmacological treatments: Sunitinib (LC laboratories, MA), DC-101 (Bio X cell, NH) and Axitinib (LC laboratories, MA) were administered to the Tg2576 mice and their WT littermates. Control groups containing both Tg2576 and WT animals were given the vehicle (PBS+DMSO) alone. Sunitinib at a dose of 80 mg/kg or Axitinib at a dose of 10 mg/kg (both dissolved in 100% DMSO at 30 mg/ml solubility and 40 mg/ml solubility respectively and diluted to the desired concentration in PBS) or the vehicle were administered to the mice via oral gavage. The treatment schedule was three times a week for a duration of one month. DC-101 was intraperitoneally injected at a dose of 0.8 mg/mouse twice a week for a month. After one month of treatment, the animals were tested for cognitive performance to evaluate the different aspects of memory and learning using a battery of tests: open field test, y-maze, fear conditioning and radial arm water maze, before euthanizing the mice to analyse the brain tissue.

Open Field Test: A plexiglass chamber measuring 50 cm (length)×50 cm (width)×38 cm (height) with dark coloured walls and a light source focused in the centre was used. Animals across all groups were placed one at a time in the arena. The floor of the chamber was demarcated into central and peripheral regions. The field was also calibrated in the computer software, so the camera can create physical distance data from pixel-based information. The system was connected to a black and white analog tracking camera with an RTV24 Digitizer that was placed overhead of the open field. The path travelled, and time spent in the either region of the field was tracked and recorded for a total of 5 minutes using this computer tracking system (ANY-maze, Stoelting). The test exploits the innate behaviour of ‘thigmotaxis’ where the mice tend to stay towards the shaded edges of an open field and keep away from the brighter centre. This implies that mice that have intact cognition and awareness of the potential danger in the environment will spend less time in the centre of the field. This test assesses an animal's anxiety, locomotion and exploration of a novel environment.

Spontaneous alternation (Y-maze): The test for novelty exploration using spatial and working memory was conducted using a symmetrical Y-maze with a grey steel bottom plate and grey Perspex® walls (Stoelting Co, Wood Dale, Ill.). Each arm of the Y-maze was 35 cm long, 5 cm wide, and 10 cm high, and the wall at the end of each arm was identified by a different colour: white, blue or red. The spatial acquisition phase comprised a one-day trial with the mice tracked while moving freely through the three arms of the Y maze during an 8-minute session. The movements were tracked by a computer tracking system (ANY-maze, Stoelting). The performance was gauged by the percentage of alternations that was calculated as the total number of alternations×100/(total number of arm entries−2). Alternation was defined as successive entries into the three arms on overlapping triplet sets. A high percentage of alternation was indicative of sustained cognition, as the animals must remember which arm was entered last to avoid re-entering it.

Contextual Fear Conditioning: The apparatus consisted of a transparent chamber inside an enclosure with an opening in the ceiling to allow video recordings. The chamber consisted of a steel grid floor connected to a shock generator scrambler. The test encompassed two sessions: conditioning and a context test. On the conditioning day, the mice were individually placed in the chamber and allowed to explore freely for 5 minutes during which, at the 180th second, they received a foot shock of 0.50-0.80 mA for 3 seconds through the bars of the floor. 24 hours after conditioning, the mice were individually placed back in the chamber for 4 minutes, this time with no noxious stimuli. The mice were monitored for movement and freezing behaviour was recorded using computer software (Limelight, ActiMetrics, Wilmette, Ill., USA). Exclusion criteria were set for freezing events less than 2 seconds. This test was used to determine associative working memory. We explored the animal's ability to associate an environment with a noxious event that it experienced there. When the animal is returned to the same environment, it generally will demonstrate a freezing response if it remembers and associates that environment with the shock. Freezing is a species-specific response to fear, which is defined as “the absence of movement except for respiration.” This may last for seconds to minutes depending on the strength of the aversive stimulus and whether the subject is able to recall the shock.

Radial arm water maze (RAWM): The RAWM contains eight swim paths (arms) extending out of an open central area, with an escape platform located at the end of any of four alternate arms called the ‘goal arms’. The start position and the goal arms were fixed throughout the duration of the study. The mice were individually placed at the ‘start position’ of the maze and given 60 seconds to locate one of the 4 escape platforms. With each trial, the platform that was used to escape was removed and not placed back into the maze until the end of that test day. The test was repeated until only one platform was left. Once the animal found the last platform it marked the end of the test for that day. In between each trial, the animal was removed and placed back in its heated home cage for 90 seconds to avoid hypothermia. These trials were conducted daily for a total of 5 days. The latency to reach the platform for each trial and the arm entries were recorded manually. Performance of memory and learning was gauged each day based on the average time taken to find the escape platforms and the total number of errors, i.e., Reference memory errors+Working memory errors. A reference memory error is defined as the entry into an arm which never had an escape platform and working memory error is said to be the subsequent entry into an arm where the platform had been removed in the previous trial.

Tissue preparation: After the behaviour studies, the animals were terminally anesthetized with ketamine/xylazine (100 mg/kg; 10 mg/kg) and perfused with PBS for 5 minutes at a 5 ml/minute flow rate. Brains were removed, and one hemisphere was fixed with 4% paraformaldehyde (PFA) for histology and stored at 4° C. and the other hemisphere was flash frozen for biochemical studies and stored at −80° C. until it was ready to be analyzed.

The PFA fixed hemispheres were embedded in a 4% agarose block and microtome sectioned to be used for immunofluorescence, whereas the flash frozen mouse brain hemispheres were homogenized mechanically using a douncer. The cytosolic fraction was used to detect soluble proteins. The pellet left behind after the initial centrifugation was resuspended in 2% sodium dodecyl sulfate (SDS) solution in dH2O. The supernatant from this treatment contained the membrane-bound proteins. The portion of the pellet that was not dissolved in 2% SDS contained the plaque-associated Aβ that was solubilized by 70% sulfuric acid treatment, lyophilized and resuspended in 1×PBS solution. 1× Halt protease and phosphatase inhibitor cocktail (ThermoFisher Scientific; 78440) was added to prevent protein degradation.

Immunofluorescence and Confocal imaging: The brain hemispheres that were fixed with 4% PFA overnight were transferred into PBS+0.01% sodium azide and stored at 4° C. The brains were embedded in 4% agarose, fixed onto the microtome stage and sectioned at a thickness of 50 μm. Mouse hippocampus (CA1 and DG) and cortex (entorhinal and prefrontal cortex), regions involved in learning and memory that are affected in AD, were examined. The brain sections were blocked with buffer (3% skimmed milk in PBS; 0.1% Tween-20) for 1 hour at room temperature followed by overnight incubation at 4° C. with primary antibodies against the various proteins of interest: CD105 (R&D Systems; AF1097), Aβ (BioLegend 39320) and tight junction protein, Occludin (abcam; ab31721), CD-31 (abcam; ab28364), and albumin (abcam; ab19194). For co-localization experiments, multiple antibodies were used on the sections simultaneously. The fluorophore conjugated secondary antibody incubation was done at room temperature for 1 hour. DAPI was used for nuclear counterstaining. Sections were then washed using PBS with 0.1% Tween-20 before being cover slipped with Fluoromount-G. Slides were allowed to air-dry overnight in the dark.

The image acquisition was done using the Olympus FV-10i confocal microscope with the high-resolution Olympus 60 X/1.4 oil-immersion objective lens. For 3D image data set acquisition, the excitation beam was first focused at the maximum signal intensity focal position within the brain tissue sample and the appropriate exposure times were selected to avoid pixel saturation. A series of 2D images (Z stack) were taken at a step size of 1 μm. The beginning and end of the 3D stack were set based on the signal level degradation. The Volocity software (PerkinElmer) was then used to process the series of images that were taken and generate a 3D reconstruction of the tissue.

Volume estimation was performed on the 3D image data sets recorded from four or more cortical or hypothalamus areas of brain tissue samples. In this procedure, a noise removal filter (either Gaussian or kernel size of 3×3) was used to remove the noise associated with the images. To define the boundary between the objects (for instance, blood capillaries or amyloid) and the background, the lower threshold level in the histogram was set to exclude all possible background voxel values. The sum of all the voxels above this threshold level was determined to be the volume. The total fluorescence volume (TFV) was detected in the field of interest and was integrated above the background by the software. The ratio of the TFV and the total volume of the field was used as a numerical representation of the expression of the protein of interest in a unit volume of the voxel. Co-localization of proteins was established by selecting the background as an area of interest (Volocity_user_guide, 2011). The thresholds for different channels/target proteins were set using this region of interest (ROI). Clicking a point outside the ROI gave the scatter plot for the entire image (Volocity_user_guide, 2011). Thresholded Pearson's Correlation coefficient (PCC) was calculated by the software (Volocity_user_guide, 2011). The closer the value of PCC is to 1, the stronger the statistical correlation is between the two targets.

Semi-quantitative analysis of TJ morphology: Brain sections were processed for immunofluorescence by staining for co-localized CD-31 and occludin proteins. In the hippocampus and cortex, individual vessels stained with anti-CD31 were scored as either normal (1) or abnormal (0) for occludin expression. Normal occludin expression was defined by observation of strong, continuous, intense and linear staining. In contrast, abnormal occludin expression was judged as weak, punctate and/or discontinuous staining. To minimize the recording of incomplete or undulating vessels as abnormal due to observed “gaps” in occludin staining, evidence of vessel continuity was sought in the images with the help of CD31 staining. The percentage of TJ disruption in a given region of the brain was defined as the percentage of blood vessels that display abnormal TJ morphology. The image acquisition and analysis were done as described above.

Blood Brain Barrier Permeability Assay: Drug- and vehicle-treated mice (n=3) were weighed and intraperitoneally (i.p.) injected with 50 μg Evans blue dye (in PBS; Sigma-Aldrich #E2129) per gram weight of the mouse (Ujiie et al., 2003). Three hours after injection, the mice were terminally anesthetized with ketamine (100 mg/kg i.p.; Narketan, Vetoquinol) and xylazine (10 mg/kg i.p.; Rompun, Bayer) and transcardially perfused with PBS for 5 minutes at 5 ml/minute flow rate (Ujiie et al., 2003). After removing the cerebellum and olfactory bulbs, the brains were weighed. The Evans blue dye was extracted through the following process; 1 mL of 50% trichloroacetic acid was added to the brain, and the samples were Dounce homogenized (pulling the plunger up and down 10 times). The homogenates were centrifuged at 13,000 rpm for 10 minutes and the supernatant diluted 1:4 with 100% ethanol. The supernatant with ethanol was read with an ELISA plate reader (Spectra Max 190; Molecular Devices, Sunnyvale, Calif.) at 620 nm to determine the optical density of the Evans Blue. The readings were divided by the weight of the brain, and the data statistically analyzed with unpaired t-tests. Evans Blue is a dye that has a high affinity for serum albumin (Ujiie et al., 2003). An intact BBB is impermeable to serum albumin and thus the injected Evans Blue remains bound to the serum albumin and does not stain the neuronal tissue. When the BBB has been compromised, albumin-bound Evans Blue enters the CNS and stains it blue, allowing for visual qualitative confirmation in addition to the fluorescent quantitative immunohistological assay.

Proximity Ligation Assay (PLA): HBEC-5i cells were seeded into culture slides in 24 well plates (75,000 cells/well) in 500 μL of DMEM media supplemented with 10% fetal bovine serum. After 24 h, cells were washed with PBS and treated with 10 uM of Aβ peptide in serum free DMEM media. The next day after treatment, cells were washed twice with PBS and fixed in 4% PFA. For PLA analysis, cells were blocked with 3% BSA, permeabilized with triton-X, 0.5% and incubated with 1/500 dilution of Anti-Angiopoietin 2 (ab8452, abcam), 1/200 dilution of anti-β-Amyloid (#39377, abcam), 1/500 dilution of anti-β-Amyloid (#93320, Biolegend), 1/200 of anti-tie2 (#05-584, Millipore) or 1/200 of Anti-VEGF Receptor 2 antibody (ab11939, abcam) for overnight at 4 degree. Protein-protein interactions were analyzed using Duolink-based in situ PLA (Sigma-Aldrich), according to manufacturer's instructions. Signals were quantified using a confocal microscope and the Duolink Image Tool. Statistical analysis was performed on GraphPad Prism using Kruskal wallis test followed by Tukey's multiple comparison (*p<0.05; **p<0.01; ***p<0.001).

Fold change of phospho-kinase levels and angiogenesis protein levels in Tg2576 mice was calculated by normalizing the mean pixel density with the WT (B6/SJL) mice.

Phospho-Kinase and Angiogenesis Proteome Arrays

Total brain tissue homogenates were prepared as described above. Homogenates were incubated on nitrocellulose membranes containing different antibodies printed in duplicates provided with Proteome Profiler Human Phospho-Kinase Array Kit (R&D Systems; ARY003B) and Proteome Profiler Mouse Angiogenesis Array Kit (R&D Systems; ARY015). The Proteome Profiler Human Phospho-Kinase Array Kit (R&D Systems; ARY003B) can also be used for mouse samples and has previously been tested by [205, 206] [127, 128]. The phospho-kinase analysis and angiogenesis analysis were performed according to the manufacturer's instructions. Phospho-kinase levels and levels of proteins involved in angiogenesis in Tg2576 mice versus the WT littermates were depicted as fold change.

FACS Analysis of Lymphocytes

Blood draws were performed on the mice and collected in EDTA-coated tubes. Lymphocytes were isolated after RBC lysis and incubated with antibodies against CD45.2 and CD45.1 in FACS buffer. The samples were analysed for CD 45 type.

Statistical Analysis: The data are presented as the mean±standard deviation. The statistical analyses were done with the help of the GraphPad Prism software using unpaired students t test when comparing two groups and a 2-way ANOVA test for multiple comparisons with a Bonferroni's test to correct for the multiple comparisons. The sample size for each experiment is indicated in the figure legend.

### Example 1

### INTRODUCTION

Growing evidence supports the concept that, in addition to neurons, the neurovascular unit is affected in AD (Biron et al., 2011; Claassen and Zhang, 2011; Pfeifer et al., 2002) and that AD may be mediated by pathological angiogenesis (Biron et al., 2011), (Chaahat Singh, 2017), (Jefferies et al., 2013). Vascular dysfunction now appears to be a crucial pathological hallmark of AD, and two key precursors to neurodegenerative changes and Aβ deposition in AD are blood brain barrier (BBB) breakdown and cerebral blood flow (CBF) impairment (Desai et al., 2009; Ujiie et al., 2003; Vagnucci and Li, 2003). These observations have led to an alternative hypothesis where angiogenesis caused by amyloidogenesis may lead to defective neuro-vasculature, thereby disrupting the BBB, impairing CBF, compromising the clearance of Aβ (Jefferies et al., 2013). Being vasculotropic, in this model, Aβ promotes more vascular pathology and hence the vicious cycle of abnormal angiogenesis underpins the disease. (Biron et al., 2013; Biron et al., 2011; Desai et al., 2009; Dickstein et al., 2006; Iadecola, 2004; Ruitenberg et al., 2005; Ujiie et al., 2003).

CBF plays a pivotal role in influencing the permeability of the BBB, and severe reductions in CBF have been seen in elderly individuals at high risk for cognitive decline and AD. Impaired CBF and compromised BBB results in the accumulation of potentially neurotoxic blood products from peripheral circulation in the brain (Dickstein et al., 2006; Ujiie et al., 2003; Zlokovic, 2008). Furthermore, physical breakdown of the BBB during angiogenesis results in disrupted tight junctions (TJs) or adherens junctions, reduction of pericytes and capillary basement membrane degradation (Claassen and Zhang, 2011; Zlokovic, 2011).

Consistent with emerging pathogenic features of the BBB in AD, we proposed a new testable paradigm for integrating vascular remodelling with the pathophysiology observed in AD in which counteracting the hypervascularity caused by neoangiogenesis might help restore the physiological state of the brain. Angiogenesis is potentiated in both AD clinical samples and in animal models of AD (Biron et al., 2013; Biron et al., 2011). To delve into the mechanism of vascular pathology in AD, we need to understand how the normal physiological process of angiogenesis becomes pathogenic. The adult vasculature is derived from a network of blood vessels created in the embryo by vasculogenesis (Bell et al., 2010). The endothelial cell lattice that is created serves as a scaffold for angiogenesis, forming the primary capillary plexus which is remodeled by the sprouting and branching of new vessels from pre-existing ones in the process of angiogenesis (Papetti and Herman, 2002). Angiogenesis occurs in the adult during physiological repair processes such as wound healing. The major factors modulating angiogenesis in the brain are the tyrosine kinase with immunoglobulin-like and EGF-like domains-2 receptor (Tie-2) (Teichert et al., 2017) and the vascular endothelial growth factor receptor 2 (VEGFR2) (Matsumoto et al., 2005), (Shalaby et al., 1995), (Sakurai et al., 2005). Vessel formation initiates with the activation of receptor Tie-2, leading to the removal of pericytes and causing vessel destabilization. The endothelial cells proliferate by activation of VEGFR2, and, as a result, the vessel starts sprouting (Papetti and Herman, 2002).

Tie 2 has two ligands, Angiopoietin-1 (Ang-1) when the vessel is stable, and Angiopoietin-2 (Ang-2) when the vessel is destabilized. In the presence of excess VEGF, inflammatory cytokines (TNFα) and hypoxia, Tie-2 preferentially binds to Angiopoietin-2. Studies have shown that Ang-1 promotes strong activation of the Tie2/PI3K/AKT pathway in quiescent, mature vessels. When the Ang-1 is present, Ang-2 is a Tie-2 antagonist; however, when Ang-1 is absent, Ang-2 acts as a TIE2 agonist, though to a weaker extent than Ang-1 (Yuan et al., 2009). When Ang-1/Tie-2 signalling is weak, AKT level is reduced and transcription factor FOXO1 is activated, which increases Ang-2 expression. Ang-2 increases the phosphorylation of Tie-2, thereby compensating for the absence of a strong Ang-1 signal (Thurston and Daly, 2012). Ang-2 is known to promote tumour angiogenesis via activation of Tie-2. While both ligands bind to Tie-2, Ang-1 binding to Tie-2 is altered by Tie-1. In the presence of the Tie-1 ectodomain, Ang-1 is unable to bind to Tie-2 (Hansen et al., 2010). However, the binding of Ang-2 to Tie-2 is unhindered by the presence or absence of Tie-1 (Hansen et al., 2010).

Ang-2, although thought to be a Tie-2 antagonist, has recently been shown to play a more nuanced role in activating angiogenesis. Hypoxia, inflammatory cytokines and VEGFA regulate the interaction of Ang-2 with Tie-2. Ang2: Tie2 binding promotes destabilization of vessels and initiates neovascularization (Thurston and Daly, 2012). Increased VEGFA enables Ang-2 to promote endothelial cell migration and proliferation, while a lack of VEGFA leads to Ang-2 initiating endothelial cell death (Thurston and Daly, 2012). These observations lead us to test the hypothesis that a mechanism mediated by Ang-2 and Tie2 underpins elevated angiogenesis in AD and anti-cancer drugs that abate angiogenesis, can reverse AD pathology.

Results

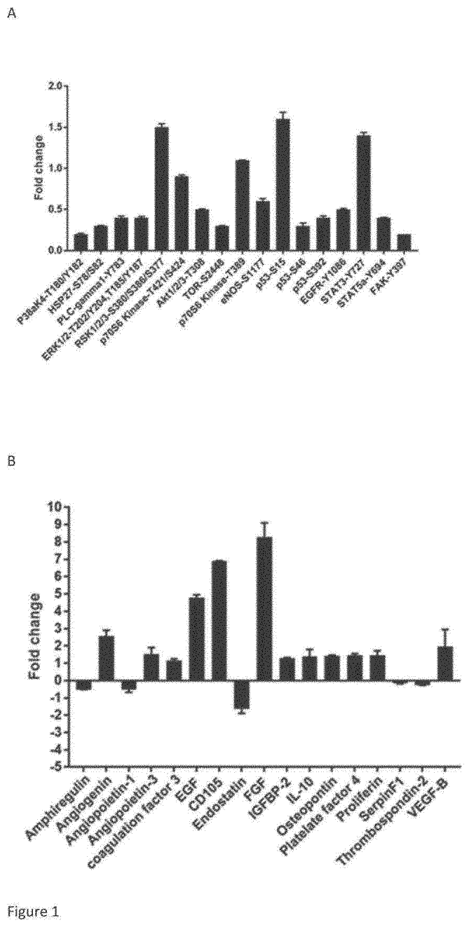

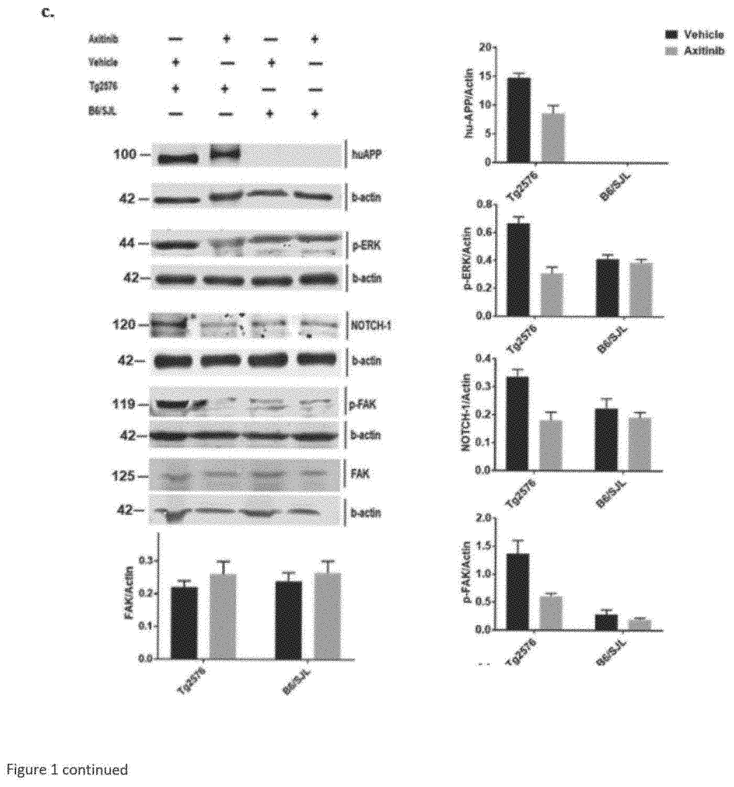

Tg2576 mice show higher expression of pro-angiogenic proteins and downstream signalling effector proteins: To investigate whether the physical interaction of Aβ with Tie-2 and its ligand Ang-2, has a functional outcome, the protein expression of downstream signalling molecules and transcription factors implicated in amyloid overproducing Tg2576 mice was analyzed. A phospho-kinase and angiogenesis proteome array analysis was conducted. The downstream signalling pathways MAPK, AKT, JAK/STAT and Wnt involved in cell proliferation, endothelial migration and cell survival were highly activated in 11 month-old untreated Tg2576 mice, as were transcription factors responsible for promoting vessel formation like STAT3, c-fos, CREB and p53 (FIG. 1a). It was observed that in comparison to untreated WT littermates, the untreated Tg2576 mice showed upregulation of CD105, vascular endothelial growth factor (VEGF), fibroblast growth factor (FGF), Osteopontin, proliferation, platelet factor 4, angiogenin and IL-10, among other proangiogenic factors (FIG. 1b). The Tie-2 receptor ligand, Ang-1, was reduced in Tg2576 mice.

Western blot analysis was done on 11-month-old Tg2576 mice and WT littermates that were treated with either vehicle or Axitinib to look at various angiogenic effectors. It was noted (FIG. 1c) that in the brains of vehicle-treated Tg2576 animals overexpressing Aβ, there were increases in APP, pERK-1, NOTCH-1 and p-FAK and that the expression of these effectors was lower in the Axitinib-treated Tg2576 mice.

Human brain endothelial cells (HBEC-5i) treated with amyloid-beta demonstrate an increase in production and activity of VEGFR2 and Tie-2 receptors that are key initiators of angiogenesis: Human brain endothelial cells were grown to confluency and treated with either PBS, Aβ1-42 peptide, Aβ1-16 peptide, Ang-2 peptide or VEGFA-165 peptide for 24 hrs. The cell lysates were tested by western blot analysis for different proteins of interest.

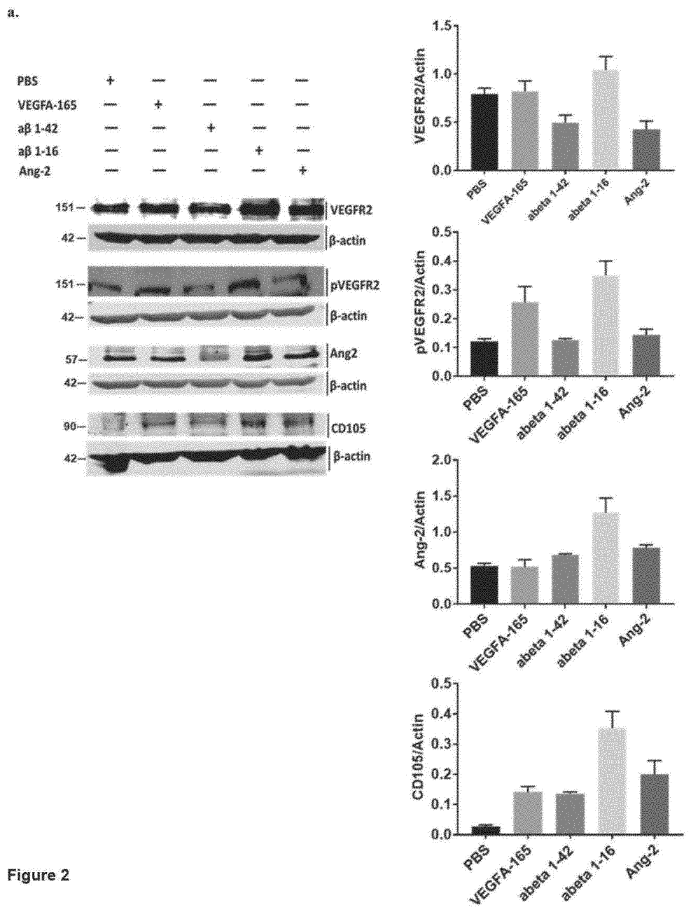

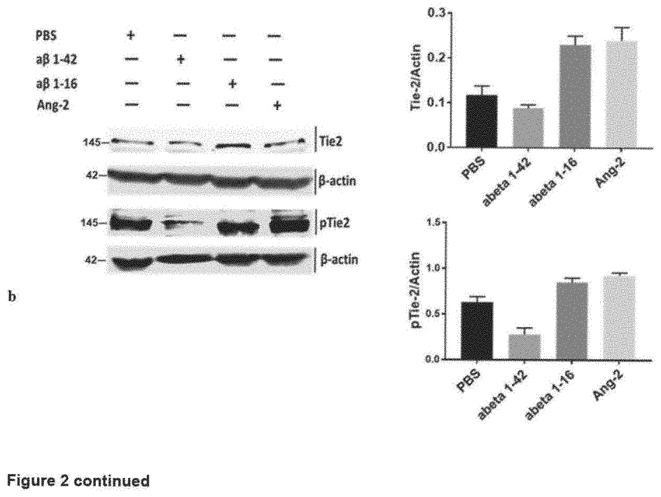

There was an increase in the expression of VEGFR2 when the cells were treated with the Aβ1-16 peptide as compared to the control (PBS-treated) cells. Aβ1-42 peptide did not exert a similar effect on the cells. There was a slight increase in the VEGFR2 expression when the cells were treated with VEGFA (the receptor-ligand) and Ang-2 peptide but not to the level achieved with the Aβ1-16 peptide. Phosphorylated VEGFR2 was also increased after Aβ1-16 peptide treatment as compared to the other treatments (FIG. 2a). Similarly, the Tie-2 receptor was upregulated after treatment with the Aβ1-16 peptide as compared cells treated with PBS or to cells treated with the Aβ1-42 peptide. Ang-2 is a Tie-2 receptor ligand that initiates a dysregulated activity of Tie-2 (Hansen et al., 2010; Thurston and Daly, 2012; Yuan et al., 2009). Aβ1-16 peptide treatment demonstrated upregulation of Ang-2 more than the control cells or Aβ1-42 peptide-treated cells (FIG. 2b).

Brains of Alzheimer's patients show co-localization of Aβ & Tie-2 and Ang-2, Tie-2 & CD105 in the cerebellum: Alzheimer's disease and normal human brain sections were stained and analyzed. We see the co-localization of Tie-2 with Ang-2 and CD105 (FIG. 3a) in the cerebellum of the AD brains. In the proximity ligation analysis (FIG. 3b), we see the interaction between Tie-2 with Aβ. We also show in FIGS. 2 and 3 that in the presence of Aβ there is increase in the expression of the neoangiogenic marker CD-105 and Ang-2, a ligand of Tie-2, is highly expressed. This was corroborated in the human AD brain sections as well. We also observed in FIG. 3c, that the AD brain sections have a discontinuous occludin tight junction protein expression pattern suggesting the presence of a disrupted BBB. These data indicate the importance of the Aβ triggered Ang-2 mediated destabilized activation of Tie-2 leading to a hyper vascular state and a disrupted BBB in the Alzheimer's brain.

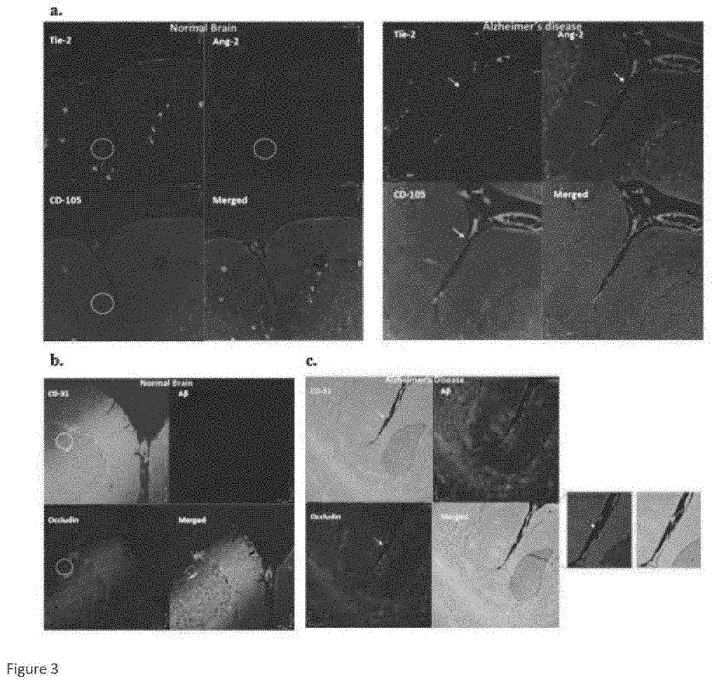

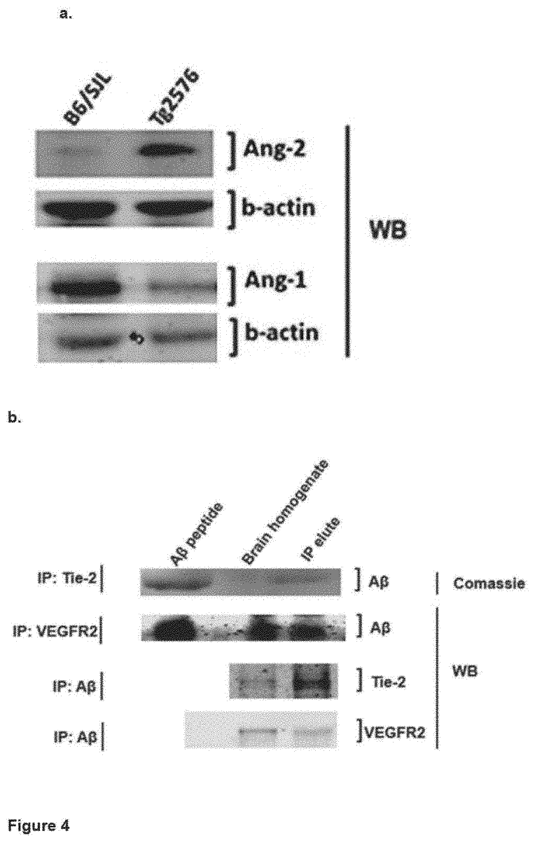

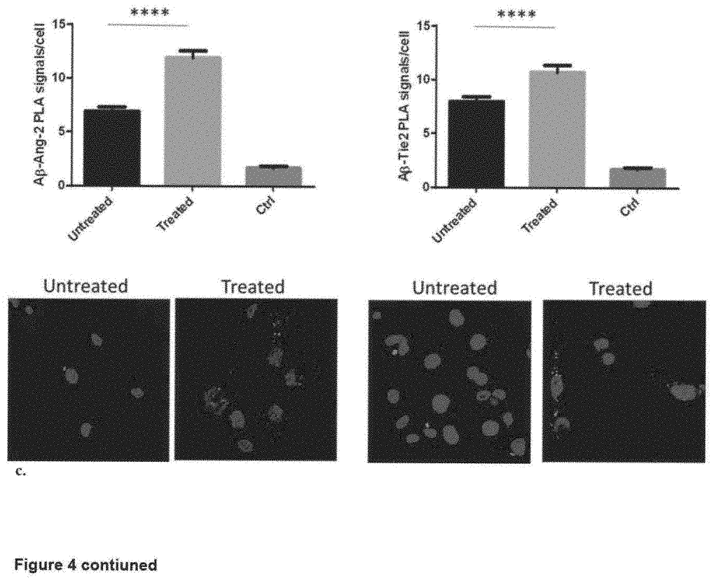

Demonstration of physically interacts between Amyloid angiogenic initiator receptor Tie-2: Western blot analysis undertaken to assess the expression levels of the two ligands, Ang-1 and Ang-2, of the angiogenesis initiator receptor Tie2. It was shown that (FIG. 4a) there was a lower expression of Ang-1 and a higher expression of Ang-2 in the 11-month-old vehicle-treated Tg2576 brain homogenates as compared to the vehicle-treated WT (B6/SJL) littermates. This indicated the occurrence of Ang-2 mediated activation of angiogenesis with the overproduction of Aβ in Tg2576 mice. To analyse this, using the homogenates from the Tg2576 mice that showed higher Ang-2 expression, co-immunoprecipitation of Aβ with Tie-2 and VEGFR2 was performed (FIG. 4b) which established a direct interaction of Tie-2 and VEGFR2 with Aβ. To confirm this, a more physiologically relevant Proximity ligation assay was performed. Using the PLA assay a significant interaction between Aβ and Tie-2, and Aβ and Ang-2 (FIG. 4c) was seen in living HBEC-5i cells treated with Aβ 1-16 peptide compared to cells treated with PBS (negative control).

Repurposing an Anti-cancer drug for the treatment of AD: Having demonstrated the mechanism underlying Tie-2 angiogenesis in AD patients and animal models of AD we sought to test whether the anti-angiogenic drug, Axitinib abates cerebral vessel growth and reverses AD pathology in the Tg2576 mouse model. Axitinib is a tyrosine kinase (TK) inhibitor of VEGFR 1 through 3. It is a second-generation TK inhibitor that has 50-450 times more inhibition potency than first-generation inhibitors like Sunitinib (Sutent; Pfizer), which is a multi-targeted TK inhibitor. Axitinib (Inlyta; Pfizer), which is approved for use in renal cell cancer in the USA and Canada, Europe, the UK and Australia (BC_Cancer_Agency, 2015; Wikipedia_contributors, 2018), has been shown to block angiogenesis, tumour growth and metastasis. We explore the ability of Axitinib to alleviate the pathogenic cerebrovascular activation, disrupted BBB and impaired cognitive function that is associated with the Swedish-familial AD mouse model, Tg2576 (Hsiao et al., 1996).

The Tg2576 AD-mouse model has been well characterized for the development of plaques at 9-months of age and cognitive decline starting at 6 months of age and progressing until the animal's death (Hsiao et al., 1996). A previous study showed that the vascular pathology and BBB disruption at 4 months of age preceded the formation of plaques (Biron et al., 2011). We chose to use 10-month old Tg2576 mice for these studies as they would, by this age, be expected already to have substantial vascular pathology (Gama Sosa et al., 2010), BBB disruption (Biron et al., 2011; Kook et al., 2013), amyloid load and cognitive decline (Kawarabayashi et al., 2001; Stewart et al., 2011; Westerman et al., 2002).

Axitinib treatment maintains the cognitive status of the Tg2576 AD mice: To assess the caution and awareness shown by mice in a novel open arena, an open field test was performed on the transgenic Tg2576 AD model mice and their wildtype (WT) littermates which were treated with either Axitinib or vehicle alone (PBS+DMSO) thrice weekly for one month at disease onset. Both male and female mice at 10 months of age were used. No differences were seen between male and female mice in the behavioural testing or their responses to the treatments, so the data from males and females were combined. Data from 3 different mouse trials and were pooled for statistical analysis (WT vehicle-treated and WT Axitinib-treated, n=15 each; Tg2576 vehicle-treated and Tg2576 Axitinib-treated, n=20 each).

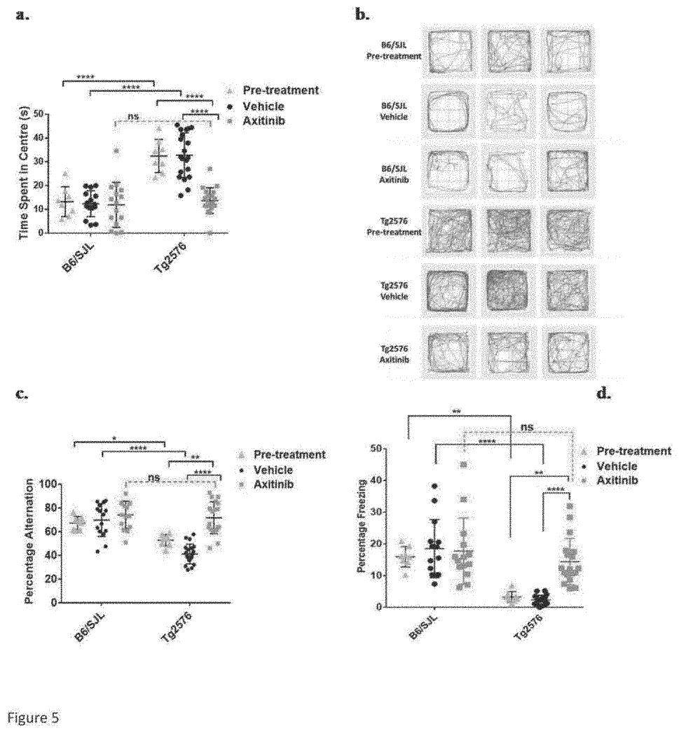

FIG. 5a demonstrates that the time spent in the centre of the field, FIG. 5b demonstrates that representative track plots of the mice from the different groups in the open field test. Inherent to their nature, it was observed that the pre-treatment and vehicle-treated-WT mice (B6/SJL) spent a majority of the time confined to the peripheral region of the field and rarely explored the centre of the open field. The results were similar in the Axitinib-treated WT mice. On the other hand, the pre-treatment and the vehicle-treated Tg2576 mice explored the entire field indiscriminately, with more distance travelled compared to all the other groups and significantly more time in the centre of the field compared to their respective WT littermates. Interestingly, after treatment with Axitinib, the Tg2576 mice explored the field to a lesser extent, preferred to move along the wall instead of into the open central area, and spent significantly less time in the centre as compared to the pre-treatment and vehicle-treated Tg2576 mice, implying that the Axitinib-treated Tg2576 were more aware of their surroundings and cautious in an open field than vehicle-treated Tg2576 animals.

Spatial and working memory assessment was done using a spontaneous alternation test (Y-maze). Pre-treatment and vehicle-treated WT mice showed an alternation of 67±5.5% (mean±standard deviation) and 70±13.8% respectively, with no significant change seen when treated with the drug (FIG. 5c). In contrast, pre-treatment and vehicle-treated Tg2576 mice showed poor performance on the test compared to their respective WT littermates, with a significantly lower percentage of alternation 52.8±4.7% and 41.2±8.3% respectively. Axitinib-treated Tg2576 mice showed significantly more alteration than pre-treatment and vehicle-treated Tg2576 mice, a performance that was indistinguishable from WT mice.

In contextual fear conditioning for the associative memory assessment, normal mice are expected to “freeze” (remain stationary) after being placed in an environment where they had previously received an electric shock. Pre-treatment, vehicle-treated and Axitinib-treated WT animals (B6/SJL) exhibited good associative memory scores by showing freezing percentages of 15.9±3.2%, 18.51±9.1% and 17.74±14.4% respectively (FIG. 5d). The vehicle-treated Tg2576 mice showed much lower freezing scores (2.3±1.5%), while the scores of Axitinib-treated Tg2576 animals were similar to those of WT control animals (14.4±7.3%). There was a significant difference between the Axitinib-treated Tg2576 mice and the pre-treatment and the vehicle-treated Tg2576 mice. Evaluation of reference memory (long-term) and working memory (short-term) was done by testing mice with the Radial Arm Water Maze (RAWM).

FIG. 5e shows the total latency time whereas FIG. 5f shows the number of errors made by the mice when locating the submerged escape platform. Comparison of the number of errors and the latency time were made between the first test day and the fifth test day within each group. Significant differences in the performance on the first test day versus the fifth test day were seen in the pre-treatment WT mice, vehicle-treated WT mice and the Axitinib-treated WT mice. No significant difference was seen in the pre-treatment Tg2576 mice and the vehicle-treated Tg2576 mice on the first day compared to the fifth day. Interestingly, Axitinib-treated Tg2576 mice showed a significant difference in the latency time and the number of errors when performance was compared between the first test day and the fifth test day. The latency time and the number of errors made on the fifth test day were compared between the different groups. A significant decrease in the latency time and the number of errors between pre-treatment WT mice and pre-treatment Tg2576 mice, as well as between vehicle-treated WT and vehicle-treated Tg2576 mice was seen.

A significant difference was also seen between the pre-treatment and Axitinib-treated Tg2576 mice, as well as between the vehicle-treated and Axitinib-treated Tg2576 mice. No significant difference was observed in the latency time and the number of errors between the fifth-day performance of the Axitinib-treated WT and the Axitinib-treated Tg2576 mice. It can thus be said that over the course of 5 days, pre-treatment, vehicle-treated and Axitinib-treated WT mice, as well as the Axitinib-treated Tg2576 mice, showed cognitive learning in terms of reference memory and working memory aspects.

A significant difference was seen in the latency time and number of errors made on test day 5 between the pre-treatment WT and Tg2576 mice as well as the vehicle-treated WT and vehicle-treated Tg2576 mice. A significant difference was also observed between the pre-treatment and Axitinib-treated Tg2576 mice as well as between the vehicle-treated and Sunitinib-treated Tg2576 mice. No significant difference was observed when comparing latency time and number of errors made by the pre-treatment and vehicle-treated Tg2576 mice as well as the Sunitinib-treated WT and Tg2576 mice on test day 5.

Axitinib Treatment Reduces Cerebral Vascular Pathology, Cerebral Aβ Load and Tight Junction Disruption in Tg2576 Mice

To assess the effect of the drug on AD pathology, the brains of treated mice were analysed by semi-quantitative western blotting to look for expression of the neoangiogenic marker CD105, as well as Aβ, amyloid precursor protein (APP) and the tight junction protein, ZO1.

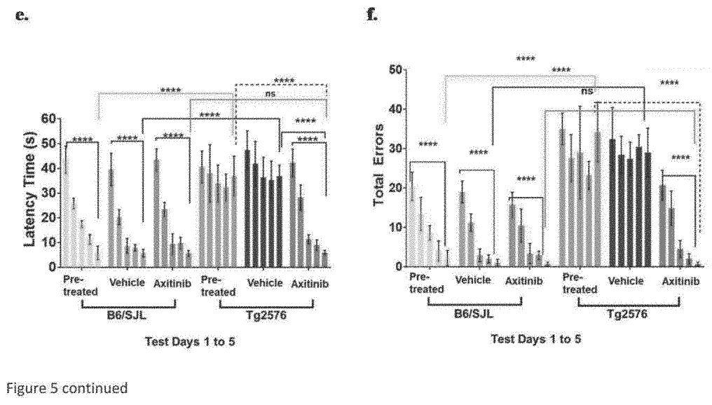

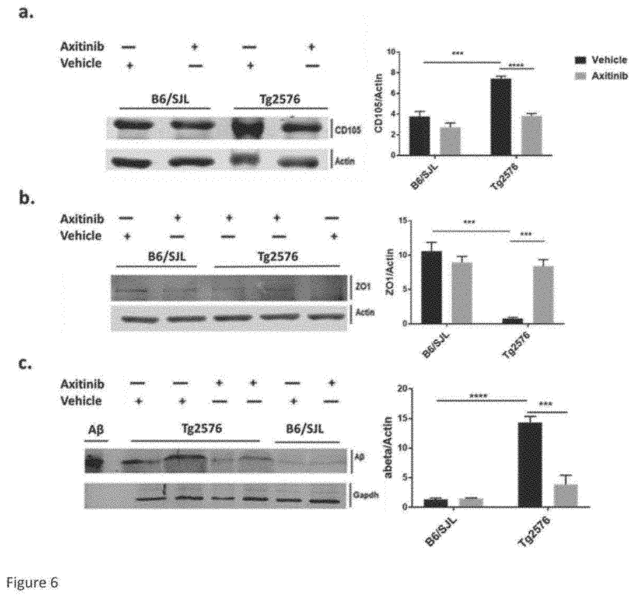

One-month treatment of the Tg2576 mice with Axitinib resulted in a significant decrease in the expression of CD105 compared to the vehicle-treated Tg2576 mice FIG. 6a). Interestingly, in Axitinib-treated Tg2576 animals, there was also a significant decrease in Aβ expression by more than one-half in comparison to the vehicle-treated Tg2576 mice (FIG. 6c). In contrast, the expression of ZO-1 was greater in Axitinib-treated Tg2576 mice compared to the vehicle-treated Tg2576 mice (FIG. 6b).

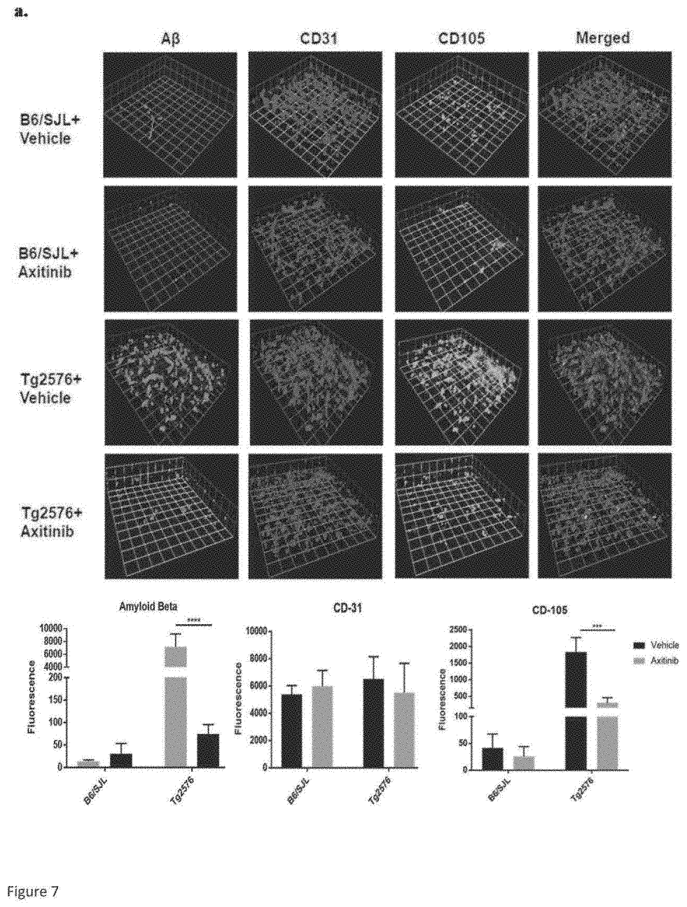

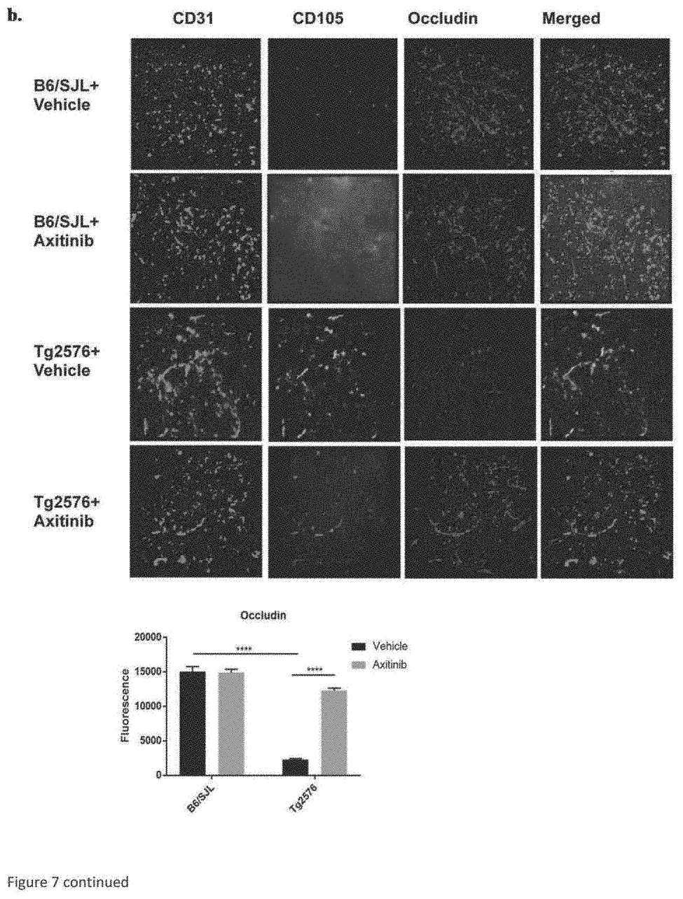

Immunofluorescence analysis of these proteins in both the cortex and hippocampus of the mouse brains confirmed the western blotting data. FIG. 7 shows representative micrographs of the cortex and hippocampus. As shown in the micrographs and histograms, amyloid staining is much heavier in the brains of vehicle-treated Tg2576 mice than in the brains of Axitinib-treated Tg2576 animals. Staining of the mature vessel marker, CD31, is similar in all the different groups; however, CD105, the sprouting vessel marker, is greater in the vehicle-treated Tg2576 group as compared to the Axitinib-treated Tg2576 or WT mice, which indicates a state of hypervascularity in the vehicle-treated Tg2576 animals (FIG. 7a). Low expression of Occludin was seen in the vehicle-treated Tg2576 as compared to the Axitinib-treated Tg2576 mice and WT (B6/SJL) animals (FIG. 7b).

Axitinib Treatment Reduces the Loss of Tight Junction Structure and BBB Integrity in Aged Tg2576 Mice

The physical seal of the BBB is maintained mainly by an intact and continuous arrangement of the tight junction proteins (TJP), ZO1, claudin and occludin, along with other components of the neurovascular unit. Their alteration can lead to disruption of the tight junctions between the endothelial cells and hence lead to increased permeability of the barrier, resulting in unhindered movement of toxic blood products into the brain, advancing AD pathology.

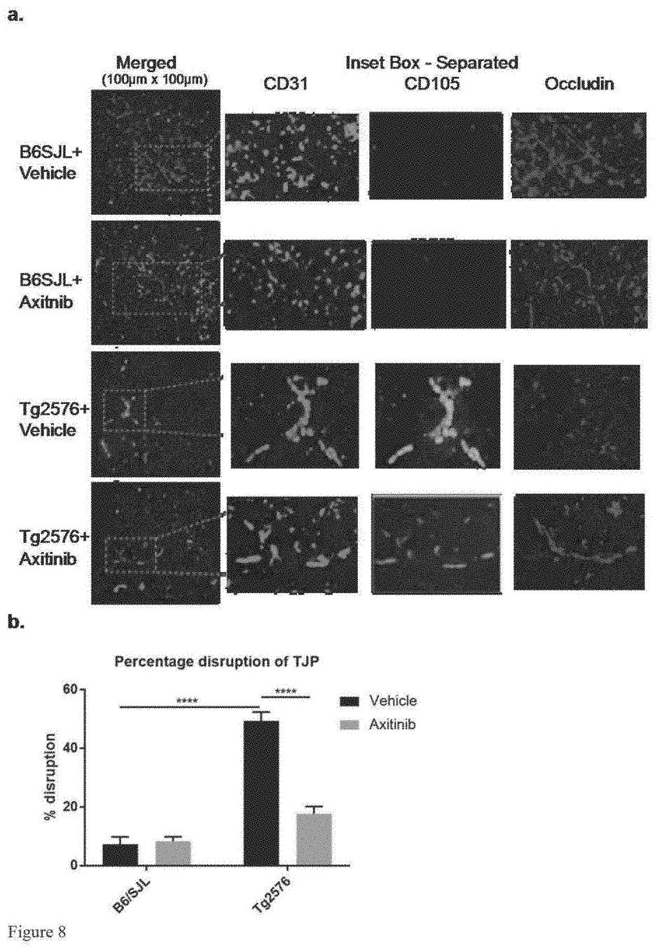

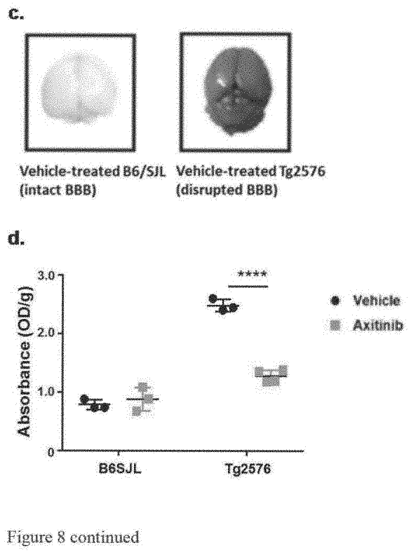

TJP disruption was analysed in the brains of Tg2576 mice and their WT littermates. A normal occludin expression pattern, as indicated by white arrows in FIG. 8a, is strong and continuous. It was observed that the WT mice showed a normal expression pattern of TJPs (as indicated with the white arrows in the micrographs) and thus a low percentage of disruption irrespective of Axitinib or vehicle treatment. In the vehicle-treated Tg2576 mice, however, there was a significant increase in the disruption of the TJPs. Tg2576 mice treated with Axitinib showed a lower percentage of the tight junction disruption, similar to the WT.

To prove that this intact arrangement of the TJP influenced the permeability of the BBB in the animals, we conducted an Evans Blue assay. Evans Blue dye binds to serum albumin, a protein to which the BBB is impermeable. A disrupted BBB allows albumin to enter the central nervous system (CNS), as indicated by the presence of the dye in the brain.

FIG. 6 shows that when Evans Blue dye is injected in mice that have an intact BBB, the dye is unable to cross into and stain the brain. FIG. 8c the WT brain looks normal, while the vehicle-treated Tg2576 brain is stained blue. These brains (cortex alone) were assessed for the extent to which the dye had crossed into the CNS. FIG. 8b. illustrates Evans blue staining measured as absorbance in terms of optical density/unit mass of the brain. Increased absorbance was noted in vehicle-treated Tg2576 mice, indicating substantial Evans Blue uptake in the brain, compared to WT littermates and Axitinib-treated Tg2576 mice.

In a separate experiment, brain sections of Tg2576 mice and WT littermates that were treated with Axitinib or vehicle were immunostained to look for the presence of albumin in the CNS. Representative micrographs of the cortical and hippocampal regions of the brain in FIG. 8d show the presence of albumin in the CNS. Minimal amounts of albumin were seen in the WT brains, indicating an intact BBB, whereas substantial staining of albumin was seen in the vehicle-treated Tg2576 mice, implying a disrupted and highly permeable BBB. The brains of the Axitinib-treated Tg2576 mice showed less albumin in the brain, demonstrating a more functional BBB.

Pathological Neo-Angiogenic Vessels Co-Localize with Pathogenic Aβ and Disrupted Tight Junction Proteins

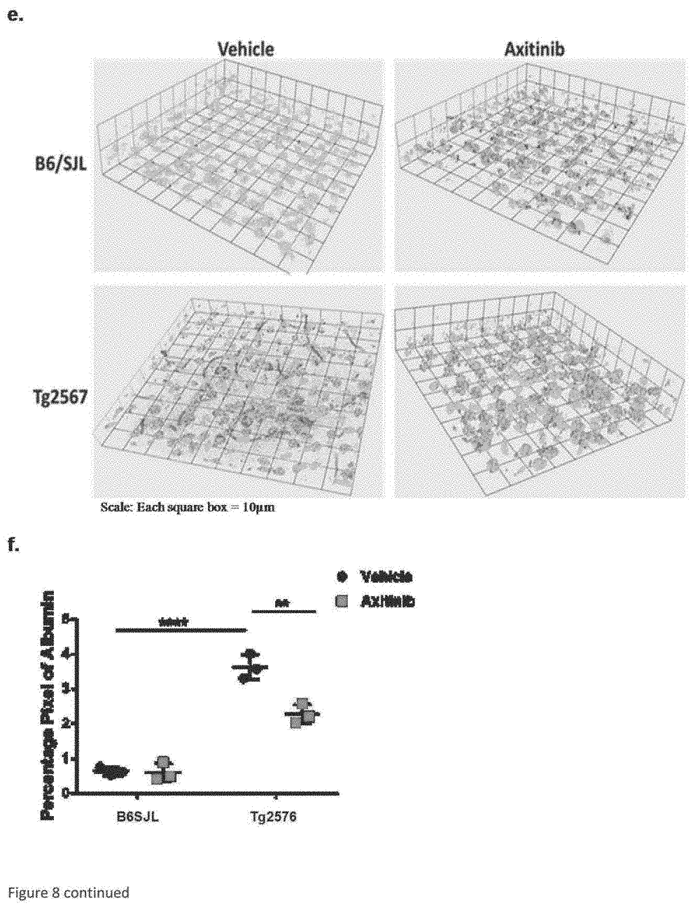

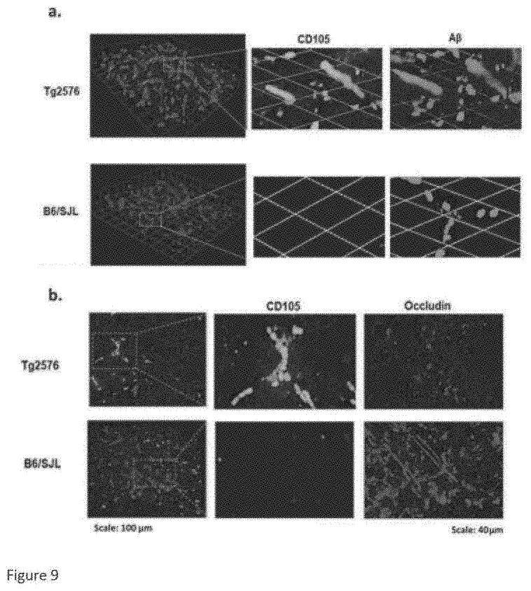

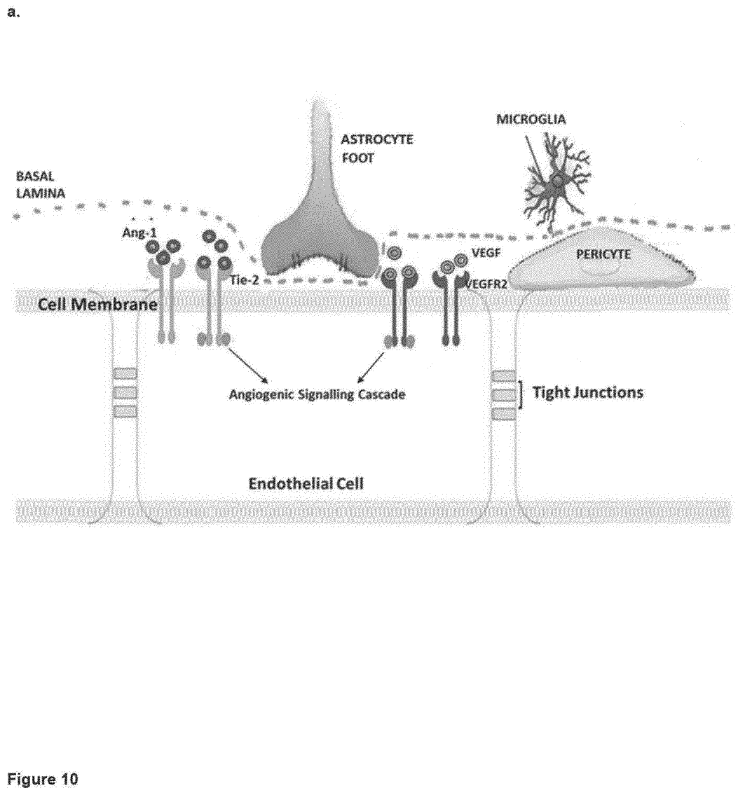

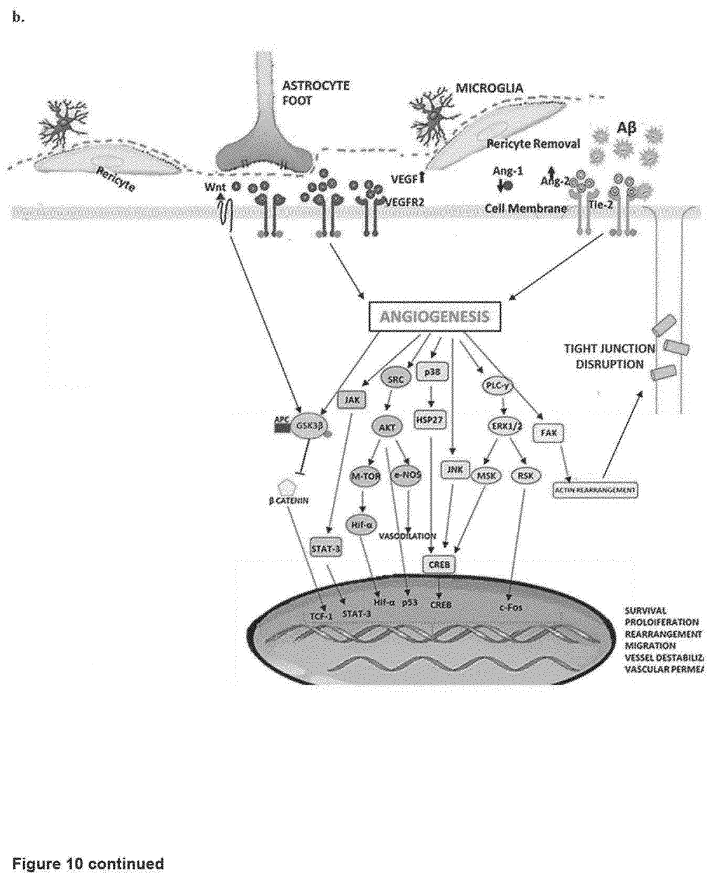

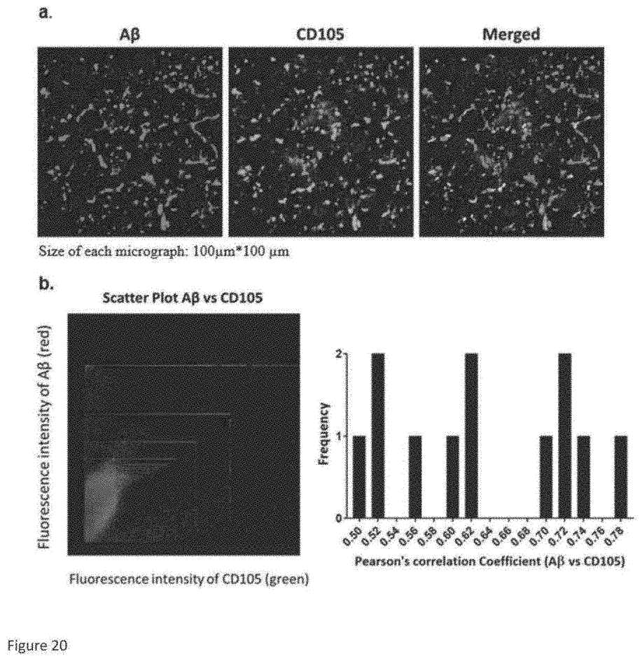

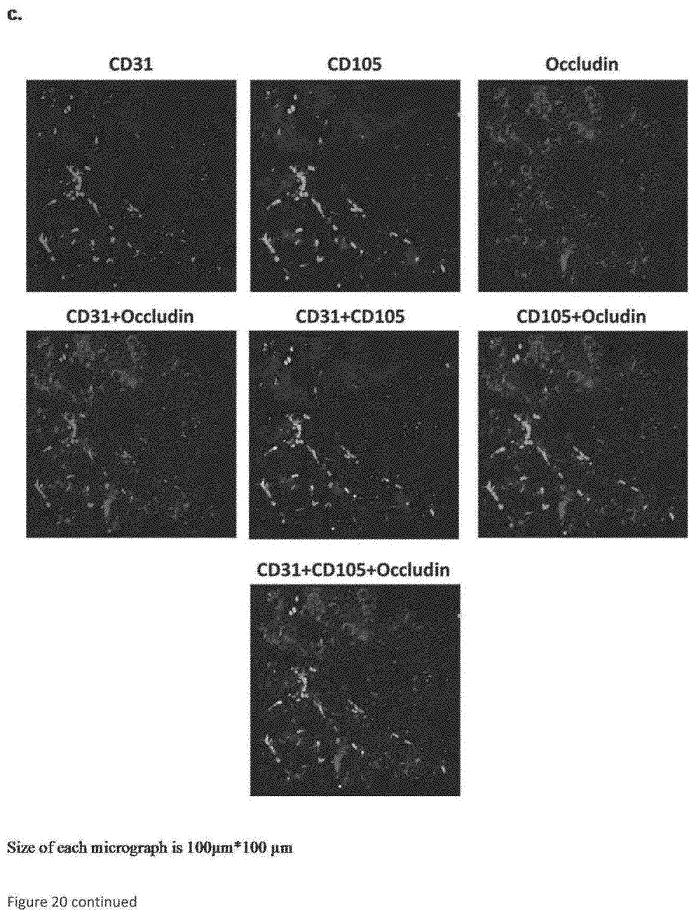

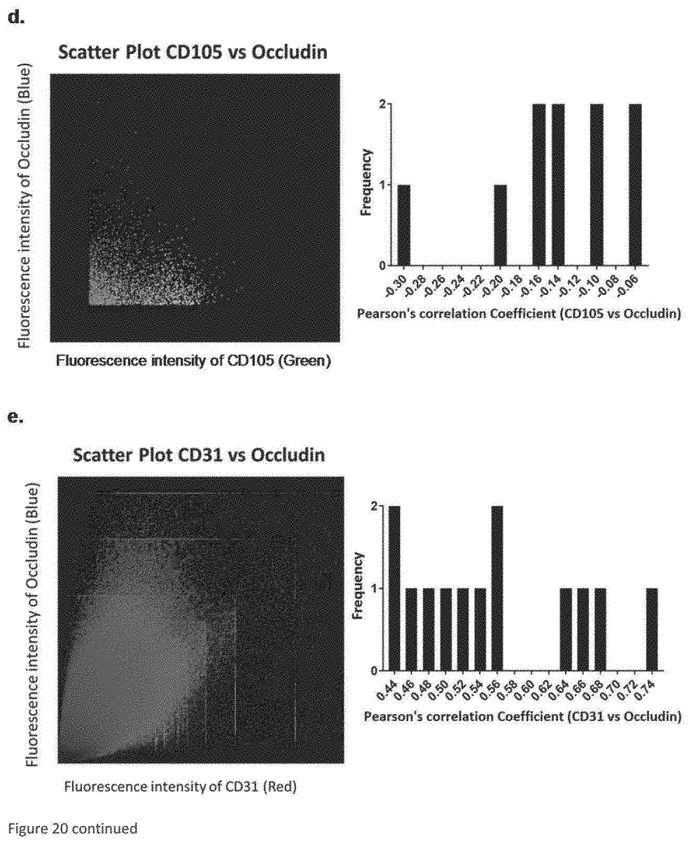

The co-localization of CD105 with Aβ and occludin is shown in FIGS. 9 and 20. Thresholded Pearson's Correlation Coefficients were calculated to assess the correlation between Aβ and CD105 expression and between CD105 and occludin expression in brain sections from vehicle-treated 11-month old Tg2576 and vehicle-treated age-matched WT littermates, where “+1” would indicate a positive correlation between the two events with an increase in one variable associated with a corresponding increase in the other, “0” would indicate no correlation, and “−1” would indicate a negative correlation with an increase in one variable associated with a corresponding decrease in the other.

A correlation coefficient of r=+0.62 (p=0.021) in vehicle-treated Tg2576 mice, which have an overproduction of Aβ, indicated a positive correlation between Aβ and CD105, suggesting a relationship between the presence of excessive amounts of amyloid and an increase in the sprouting pathogenic vessels in the cerebral vasculature. On the other hand, a correlation coefficient of r=−0.73 (p=0.006) in the vehicle-treated Tg2576 mice indicates that CD105 and occludin expression are negatively correlated. WT brains show low expression of Aβ and CD105 and have normal levels of tight junction proteins.

### DISCUSSION

This study described the Tie-2 mechanism underlying pathogenic angiogenesis of AD in humans and animals and applies this knowledge to test the efficacy of an anti-cancer drug for halting and reversing AD. Our gene expression profiling of the brains of AD Tg2576 mice demonstrate an elevation of angiogenic gene programs. Studies with cultured human brain endothelial cells (HBEC-5i) treated with amyloid-beta demonstrate an increase in production and activity of VEGFR2 and Tie-2 receptors, both key initiators of angiogenesis. In our studies, the brains of Alzheimer's patients exhibited the presence of amyloid plaques, cerebral amyloid angiopathy, hypervascularity and disrupted tight junctions. Human AD brains also show co-localization of Aβ & Tie-2 and Ang-2, Tie-2 & CD105 in the precentral gyrus and the cerebellum.

Direct binding and interaction studies using PLA methodologies demonstrate that Beta-Amyloid physically interacts with the angiogenesis initiator receptor Tie-2. A profound reduction in plaque number, the reestablishment of the BBB, restoration of cognitive function and a reversal of cerebral vessel growth was observed after treatment of 10-month-old Tg2576 mice with the anti-angiogenic, tyrosine kinase inhibitor, Axitinib for a period of one month. These discoveries provide a new entry point for treating AD and strengthens the evidence for the vascular aetiology of AD. There is a close association of AD with cerebrovascular amyloid angiopathy (CAA), in which Aβ deposition is found in pial and intracerebral vessels (Claassen and Zhang, 2011). This is observed in about 90% of AD cases. CAA gradually causes vascular smooth muscle degeneration and increased vessel stiffness, eventually altering vascular function. It may also lead to the blocking of the perivascular and BBB routes for drainage of Aβ (Claassen and Zhang, 2011). Studies indicate that pathological angiogenesis and BBB disruption occur as a compensatory response to impaired CBF (Ruitenberg et al., 2005; Vagnucci and Li, 2003). Our data explain these previous studies.

Prior to this study, it was unclear whether angiogenesis is a direct or an indirect initiator of BBB disruption in response to activation by Aβ or mechanisms such as oxidative stress and inflammation (Vagnucci and Li, 2003). Aβ-induced neuroinflammatory responses facilitate the release of angiogenic activators like VEGF and thrombin. VEGF not only stimulates angiogenesis but also affects the permeability of the blood vessels. Thrombin is known to synergize with VEGF to increase endothelial cell proliferation (2018). Thrombin induces the endothelial cells to further secrete Aβ, promoting the generation of reactive oxygen species and additional endothelial damage, causing a cycle of neurotoxic insult (Vagnucci and Li, 2003). Aβ has been implicated as a modulator of blood vessel density and vascular remodelling through angiogenic mechanisms (Biron et al., 2011).

Previous studies reported that BBB integrity in the Tg2576 mouse model is compromised by hypervascularity as early as four months of age, and this preceding the formation of plaques (Ujiie et al., 2003). It was demonstrated that TJ disruption is considerably higher in aged Tg2576 mice compared to age-matched WT littermates and young mice of both genotypes (Biron et al., 2011; Ujiie et al., 2003). In the same study, the cerebrovascular integrity of the Tg2576 mouse model was examined in conjunction with markers of angiogenesis and apoptosis. Aged Tg2576 mice, when compared to age-matched WT littermates, show a significant increase in the incidence of disrupted TJs that was directly linked to neoangiogenesis and an overall increase in microvascular density but not to apoptosis (Ujiie et al., 2003). These observations in the AD mouse model parallel those seen in AD patients when compared to control groups (Biron et al., 2011).

In this study, immunofluorescence colocalization analysis of the brains of 11-month-old vehicle-treated Tg2576 and WT mice demonstrated a positive correlation between the neoangiogenic marker, CD105 and Aβ, and a negative correlation between CD105 and the tight junction protein, occludin. This suggests that the increase of Aβ will be associated with an increase in CD105 and an increase in CD105 will be associated with a decrease in occludin expression. Upregulation of CD105, a marker of neoangiogenesis, is seen in the brains of Tg2576 mice. Increased proangiogenic signals like VEGF, Hypoxia-inducible factor-1 (Hif-α) and decreased Ang-1 are also seen in 11-month-old Tg2576 mice, either vehicle-treated or untreated, compared to age-matched vehicle-treated or untreated WT littermates. These observations are consistent with studies in which angiogenesis is upregulated in the absence of Ang-1 and/or in the presence of VEGF (Hansen et al., 2010; Thurston and Daly, 2012; Yuan et al., 2009). Since the Tg2576 mouse strain has been shown to produce excessive amounts of Aβ, we can say that Aβ itself has a role in the activation of the angiogenesis by either interacting with VEGFR-2 and/or Tie-2, recruiting VEGFA to activate VEGFR2, and mediating Ang-2-dependent activation of Tie2, ultimately leading to dysregulated neovascularization.

A model is shown in FIG. 9: Angiogenesis is positively correlated with amyloid beta and negatively correlated with tight junction proteins. Micrographs representing hippocampal and cortical co-localization of a) CD105 (green), Aβ (red) and CD31 (blue) b) CD105 (green), occludin (blue) and CD31 (red). The first column represents the micrographs showing the brain stained with a combination of antibody staining. The white overlay indicates the area in the field of view that was magnified and shown in the next two columns labeled either CD105 and Aβ or CD105 and Occludin. Comparing the brain sections from 11-month-old vehicle-treated Tg2576 mice and vehicle-treated WT littermates using total fluorescence volume, the Thresholded Pearson's Correlation Coefficient was calculated to assess the correlation between Aβ and neo-angiogenic marker, CD105 and between neo-angiogenic marker, CD105 and tight junction protein, Occludin. This indicated that in Tg2576 mice, a positive correlation coefficient of r=+0.62 was seen between Aβ and CD105 and a negative correlation coefficient of r=−0.73 was seen between CD105 and occludin.

Figure to explain how amyloid may initiate pathogenic angiogenesis and lead to the disruption of the BBB, a gateway to pathologies seen in AD. Increased levels of Aβ interacting with cerebral vessels in Tg2576 mouse brains in the presence of low levels of Ang-1 lead to recruitment of VEGF in the vicinity of the Tie-2 receptor. This assists Ang-2 to bind to Tie-2 and activates destabilized vessel formation, indicated by the increase in the downstream signalling pathways and activation of pro-angiogenic transcription factors for endothelial survival, proliferation, migration and production of VEGF. This VEGF effector moves out into the extracellular space, further facilitating Ang-2 in continuously binding to Tie-2 receptor molecules and constitutively activating endothelial cells to form pathogenically sprouting vessels. An increase in levels of the effector molecule, Focal Adhesion Kinase (FAK), leads to rearrangement of the actin cytoskeleton that maintains the physical intactness of the tight junctions and thus destabilizes the vessels, thereby increasing vascular permeability. This allows for the toxic blood molecules to enter the CNS and cause inflammation, hypoxia and neuronal pathologies. In this way, angiogenesis would culminate in establishing AD pathology and cognitive decline.

To answer some of the questions surrounding the proposed mechanisms, human brain endothelial cells were treated with either Aβ (1-16), Aβ (1-42), which is predominantly found in plaques; Ang2; VEGFA or PBS. Aβ (1-16) is one of the more predominant types of peripheral amyloid that is produced from non-neuronal sources (FIG. 2). It was noted that Aβ (1-16)-treated cells exhibit higher expression levels and higher phosphorylation levels of VEGFR2 and Tie2 receptors and higher expression of CD105 compared to PBS-treated cells or Aβ (1-42)-treated cells. This supports the idea that it is the soluble and circulating species of Aβ, and not the plaque-associated species that initiate angiogenesis. The Aβ (1-16)-treated cells also display increased expression of Ang-2, supporting the proposed idea of Ang-2-dependent activation of dysregulated angiogenesis.

Based on the angiogenic mechanism we identified in cerebral vasculature of AD subjects, we explored the effect of a second-generation anti-angiogenic small molecule TKI, Axitinib, that targets the VEGFR family and also attempt to understand the molecular mechanism of how amyloidogenesis leads to activation of pathological angiogenesis causing the breakdown of the BBB. 10-month old pre-treatment Tg2576 mice and WT littermates along with 11-month old Axitinib and vehicle-treated Tg2576 mice and WT littermates were tested for different aspects of cognition. WT mice, as well as Axitinib, treated Tg2576 mice showed significantly higher cognition compared to the pre-treatment and vehicle-treated Tg2576 mice in respect to spatial awareness, exploration, associative memory, working memory and reference memory. The conclusion drawn from these results is that one-month treatment with Axitinib can improve the above-stated memory aspects in aged Tg2576 mice. It was also noted that Axitinib treatment showed lower expression of angiogenic marker CD105, lower amyloid load, higher expression of TJPs like ZO1 and occludin, and a more functional BBB in aged Tg2576 mice. Thus, Axitinib can alter cerebral pathology, amyloid load and other pathological indications seen in the Tg2576 mouse model of AD and show great potential as a proof of concept that with the inhibition of cerebral neo-angiogenesis cognitive decline can be treated as well as the molecular pathology seen in AD can be altered.

This study showed that treatment with Axitinib improves cognition of the aged Tg2576 mice. Future studies of characterizing pre-treatment 10-month-old Tg2576 mice will help us address this question of whether Axitinib treatment forestalls the progression of the molecular pathology or reduces it. If the pathological changes seen in the Axitinib-treated Tg2576 mice at 11 months of age are similar to those seen in the pretreated Tg2576 mice at 10 months, then the drug will be considered preventative towards molecular pathology. However, if the Tg2576 mice demonstrate worse pathology and functional deficits before treatment than after one month of Axitinib treatment, then the drug will prove therapeutic towards molecular pathology. Similar to what was seen in the cognitive assessment, there may not be any significant difference in the molecular pathology between the 10-month old pre-treatment and the 11-month old vehicle-treated Tg2576 mice and there is a significant difference between drug-treated and vehicle-treated Tg2576 mice. Hence, it can be we can say that concerning molecular pathology, the drug treatment is likely to be therapeutic rather than simply being preventative, however further studies are needed to prove this aspect of the anti-angiogenic drug, Axitinib.

Therapies directed at the amyloid cascade pathway have thus far failed. Consequently, new approaches to prevent and treat AD are urgently needed. Vascular dysfunction now appears to be a crucial pathological hallmark of AD and the two key precursors to neurodegenerative changes and Aβ deposition result from cerebral blood flow impairment and the breakdown of the blood-brain barrier. This has spawned an alternative hypothesis of sporadic AD aetiology where impaired cerebral blood flow leads to faulty clearance of Aβ, triggering angiogenesis, breakdown of the BBB and vascular remodelling, resulting in amyloid plaques in the central nervous system, neuronal death and subsequent cognitive decline. In familial forms of AD, the initiation events would be heightened Aβ production that triggers angiogenesis, bypassing the requirement for impaired cerebral blood flow.

In conclusion, it is imperative to understand the pathophysiology of AD disease progression, and new research directions are needed. This is the first study which identifies a mechanism that overabundance of amyloid leads to activation of destabilized and leaky vessels through the Angiopoietin-2 mediated Tie-2 dysregulated cerebral angiogenesis, resulting in a cascade of downstream signalling pathways, culminating in molecular pathologies seen in AD. The therapeutic approach we describe that is directed repurposing anti-cancer drugs to modulating cerebral angiogenesis, distinguished from targeting the amyloid cascade, may prove efficacious towards AD and related vascular diseases of the brain.

### Example 2

Sunitinib Modulates Cognition to Improve Certain Aspects and Prevents Others from Declining in Aged Transgenic AD Mice—Tg2576 Mice

To assess the caution and awareness shown by mice in a novel open arena, an open field test was performed on the transgenic AD model mice, Tg2576, and their wildtype (WT) littermates treated with either Sunitinib or vehicle alone (PBS+DMSO) thrice weekly for one month at 10 months of age; both male and female mice were used for the study.

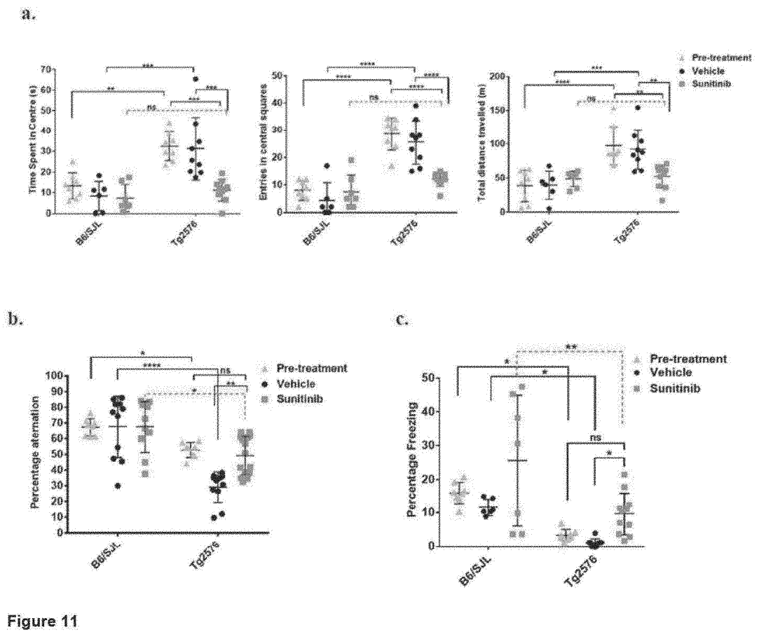

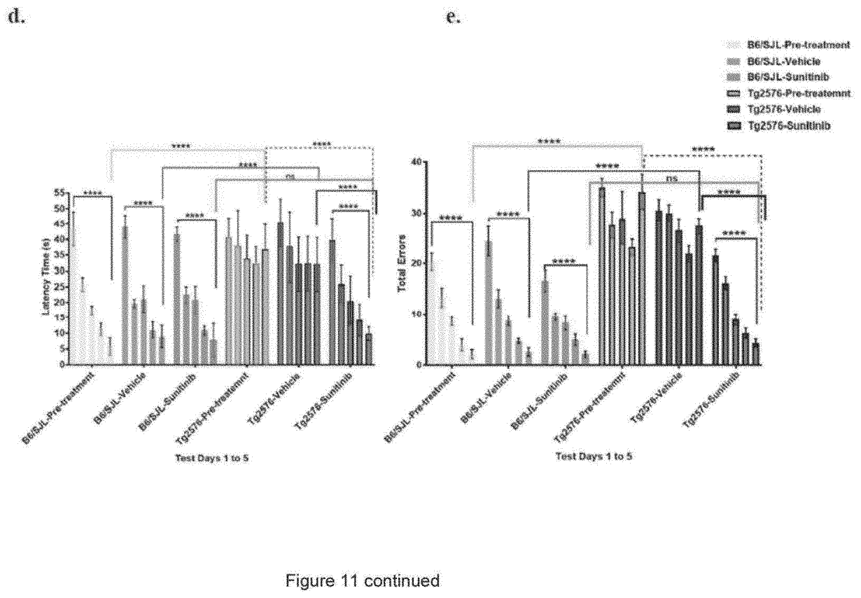

FIG. 11a. shows the time spent in the centre of the field, the number of entries made into the central region and total distance travelled by the mice. Inherent to their nature, it was observed that the pre-treatment-WT littermate mice (B6/SJL) spent significantly less time in the centre of the field as compared to the pre-treatment-Tg2576 mice at 10 months of age. Similarly, the vehicle-treated WT mice spent significantly lesser time in the centre compared to the vehicle-treated Tg2576 mice that explored the entire field indiscriminately. Sunitinib-treated WT mice also spent the majority of the time confined to the peripheral region of the field and very rarely explored the centre of the open field. After treatment with Sunitinib, Tg2576 mice spent less time in the centre with no significant difference when compared to the Sunitinib-treated WT mice. A significant difference was also seen between the Sunitinib-treated Tg2576 mice and both, the vehicle-treated and the pre-treatment Tg2576 mice. Similar outcomes were observed with the total number of entries made in the centre of the field and the total distance travelled by the mice.

Spatial and working memory assessment was done using a spontaneous alternation test (Y-maze) (FIG. 11b). Pre-treatment WT mice showed an alternation of 67.25±5.5% (mean±standard deviation) and vehicle-treated WT mice showed an alternation of 43.27±3.6% between arms; no significant change was seen when WT animals were treated with the drug. Contrastingly, pre-treatment Tg2576 mice exhibited a poor performance on the test with an alternation of 52.82±4.7%. The vehicle-treated Tg2576 mice significant lower percentage of alternation (29.1±9.7%) compared to the vehicle-treated WT mice. The Sunitinib-treated Tg2576 mice, with 49.3±12.1% alternation, show significantly higher alternation compared to the vehicle-treated Tg2576 mice, however, was not significantly different compared to the pre-treatment Tg2576 mice.

In contextual fear conditioning for the associative memory assessment, mice remained stationary after being placed in an environment where they had previously received an electric shock (FIG. 11c). Both pre-treatment and vehicle-treated WT animals (B6/SJL) exhibited higher associative memory of 15.9±3.2% and 11.5±2.5% respectively than the pre-treatment and vehicle-treated Tg2576 mice showing freezing percentages of 3.2±1.6 and 1.04±1.1% respectively. Sunitinib-treated WT mice had a significantly higher % alternation (25.4±19.4) as compared to vehicle-treated WT as well as Sunitinib-treated Tg2576 mice. Associative cognition of the Sunitinib-treated Tg2576 animals (10.9±5.8%) was significantly greater than in the vehicle-treated Tg2576 animals however it was not significantly higher than the pre-treatment Tg2576 mice.

Evaluation of reference memory (long-term) and working memory (short-term) was done by testing mice with the Radial Arm Water Maze (RAWM). FIG. 11d shows the total latency time whereas FIG. 11e shows the number of errors made by the mice when locating the submerged escape platform.

Comparison in the number of errors and the latency time were made between the first test day and the fifth test day within each group. Significant differences in the performance on the first test day versus the fifth test day were seen for the pre-treatment WT mice, vehicle-treated WT mice and the Sunitinib-treated WT mice. No significant difference was seen in the pre-treatment Tg2576 mice and the vehicle-treated Tg2576 mice on the first day compared to the fifth day. Interestingly, Sunitinib-treated Tg2576 mice showed a significant difference in the latency time and the number of errors when performance was compared between the first test day and the fifth test day.

The latency time and the number of errors made on the fifth test day were compared between the different groups. A significant decrease in the latency time and the number of errors between pre-treatment WT mice and pre-treatment Tg2576 mice, as well as between vehicle-treated WT and vehicle-treated Tg2576 mice was seen. A significant difference in the performance on the fifth day was also seen between the pre-treatment and Sunitinib-treated Tg2576 mice, as well as between the vehicle-treated and Sunitinib-treated Tg2576 mice.

No significant difference was observed in the latency time and the number of errors in the performance on the fifth day between the Sunitinib-treated WT and the Sunitinib-treated Tg2576 mice. It can thus be said that over the course of 5 days, pre-treatment, vehicle-treated and Sunitinib-treated WT mice, as well as the Sunitinib-treated Tg2576 mice, showed cognitive learning in terms of reference memory and working memory aspects.

Sunitinib Treatment Reduces Cerebral Vascular Pathology, Cerebral Aβ Load and Tight Junction Disruption in Tg2576 Mice

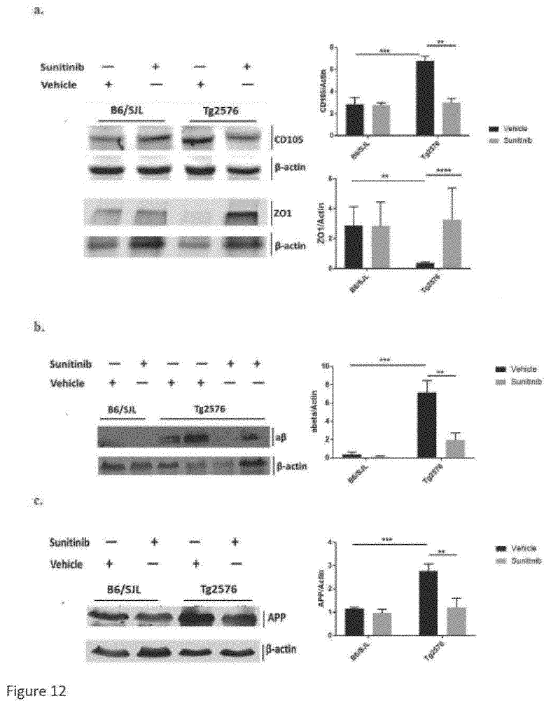

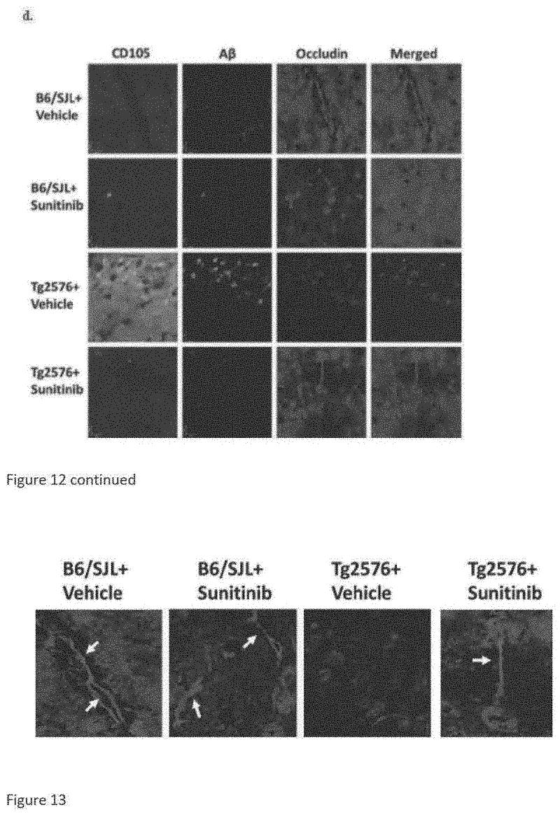

To assess the effect of the drug on AD pathology, the brains of treated mice were analysed by semi-quantitative western blotting to look for protein expression of the neoangiogenic marker CD105, as well as of Aβ, amyloid precursor protein (APP) and the tight junction protein, ZO1. One-month Sunitinib treatment of 10-month old Tg2576 mice resulted in a significant decrease in the expression of CD105 as compared to the vehicle-treated Tg2576 mice (FIG. 12a). Higher expression of ZO1 in Tg2576 mice treated with the drug compared to the vehicle-treated Tg2576 mice (FIG. 12a) indicated the presence of tight junctions in treated animals. Significantly lower Aβ expression was observed in Tg2576 mice treated with Sunitinib in comparison to vehicle-treated Tg2576 (FIG. 12b). A reduction in overall APP protein expression was also observed in Sunitinib-treated Tg2576 mice in comparison to vehicle-treated Tg2576 mice (FIG. 12c).

Immunohistological analysis of these proteins in the cortex and hippocampus of the mouse brains confirmed the western blotting data. FIG. 12d shows representative micrographs. It can be noted in the histograms that there is amyloid staining in the vehicle-treated Tg2576 mice and that amyloid is significantly less abundant in the Sunitinib-treated Tg2576 group. Expression of CD105, the sprouting vessel marker, was higher in the vehicle-treated Tg2576 group, which indicates a state of hypervascularity, as compared to the WT animals. This hyper-vascular state was not apparent when Tg2576 mice were treated with Sunitinib. Lower expression of the tight junction protein, occludin, indicative of BBB impairment, can be seen in the vehicle-treated Tg2576 as compared to B6/SJL or Sunitinib-treated Tg2576 mice (FIG. 12d).

Sunitinib Treatment Reduces the Loss of Tight Junction Structure and BBB Integrity in Aged Tg2576 Mice.

The physical seal of the BBB is maintained mainly by an intact continuous arrangement of the tight junction proteins (TJP), ZO1, claudin and occludin, along with other components of the neurovascular unit. Their alteration can lead to disruption of the tight junctions between the endothelial cells and increased permeability of the barrier, resulting in unhindered movement of damage-promoting blood products into the brain, advancing AD pathology.

The percentage the TJP disruption was analysed in Sunitinib-treated Tg2576 and control mice. WT mice showed a normal continuous expression pattern of TJPs (as indicated with the white arrows in the micrographs in FIG. 13) and thus a low percentage of disruption, irrespective of Sunitinib or vehicle treatment. In the vehicle-treated Tg2576 mice, however, there was a significant increase in the disruption of the TJPs. Tg2576 mice treated with Sunitinib showed a TJP expression pattern similar to that in the WT mice.

To prove that this intact arrangement of the TJP influenced the permeability of the BBB in the animals, we conducted an Evans Blue assay. Evans Blue dye binds to serum albumin, a protein to which the normal BBB is impermeable. A disrupted BBB allows albumin to move across and enter the central nervous system (CNS), which is indicated by the presence of the dye in the brain.

Brains (cortex and hippocampus) were assessed for the extent to which the dye had crossed into the CNS.

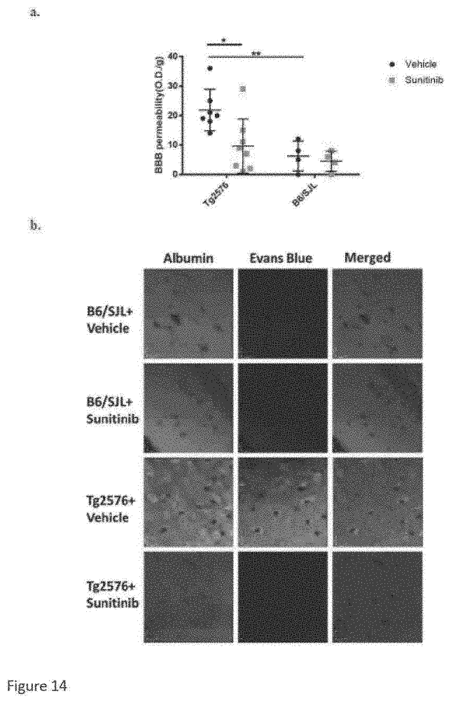

FIG. 14a. illustrates absorbance attributed to Evans blue staining in terms of optical density/unit mass of brain. A higher absorbance was noted in vehicle-treated Tg2576 mice compared to WT littermates. Lower uptake of Evans Blue into the CNS was seen after Sunitinib treatment of Tg2576 mice than in vehicle-treated Tg2576 mice.

FIG. 14b show the presence of albumin detected with the help of fluorochrome labeled antibody and the auto-fluorescing Evans blue in the CNS. Minimal amounts of albumin were seen in the WT brains, indicating an intact BBB, whereas the substantial Evans Blue staining seen in the vehicle-treated Tg2576 mice implies a disrupted and highly permeable BBB. The brains of the Sunitinib-treated Tg2576 mice show that much less albumin crosses into the CNS in comparison to the vehicle-treated mice, demonstrating a functional BBB after Sunitinib treatment.

Results for DC-101

DC-101 modulates cognition to improve certain aspects and prevents others from declining in aged transgenic AD mice-Tg2576 mice: To assess the caution and awareness shown by mice in a novel open arena, an open field test was performed on the transgenic AD model mice, Tg2576 and their wildtype (WT) littermates which were treated with either DC-101 or vehicle alone (PBS+DMSO) thrice weekly for one month at disease onset. Both male and female mice at 10 months of age were used. No differences were seen between male and female mice in the behavioural testing or in their responses to the treatments, so data for both sexes were pooled in all analyses.

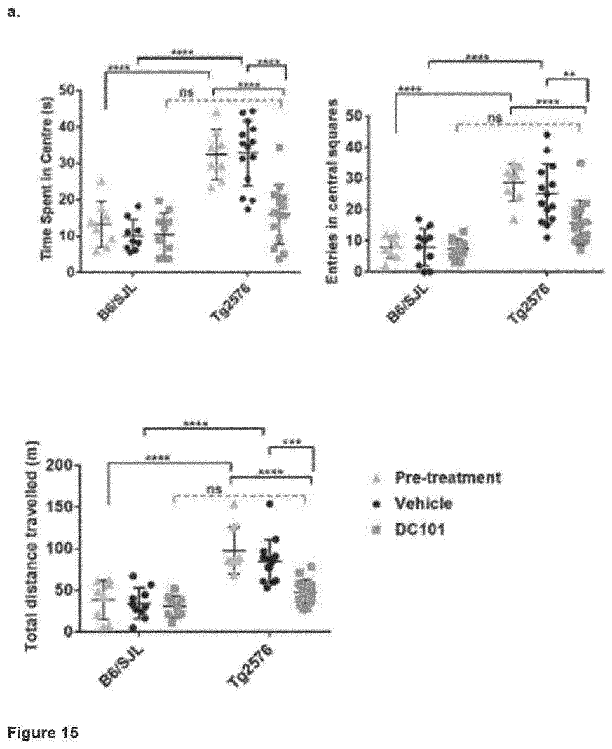

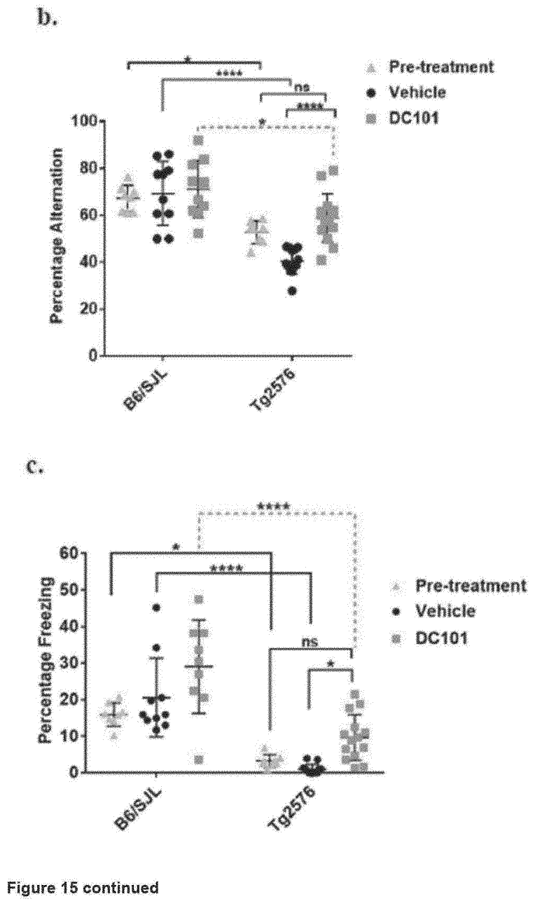

Inherent to their nature, it was observed that the WT littermate (B6/SJL) mice pre-treatment, as well as vehicle-treated spent majority of the time confined to the peripheral region of the field and very rarely, explored the centre of the open field. This did not change in the WT mice treated with DC-101 (FIG. 15a). On the other hand, the pre-treatment and vehicle-treated Tg2576 mice explored the entire field indiscriminately, with significantly more distance travelled compared to all the other groups and spent significantly more time and a higher number of entries into the centre of the field to as compared to the WT littermates. After treatment with DC-101, the Tg2576 mice explored the field to a lower extent and spent significantly less time in the centre as compared to the pre-treatment and vehicle-treated Tg2576 mice.

Spatial and working memory assessment was done using a spontaneous alternation test (Y-maze). WT mice pre-treatment and vehicle-treated showed an alternation of 65.25±5.5% and 70.1±13.6% respectively (mean±standard deviation) between arms with no significant change seen after treatment with DC-101 (71.2.6±12.1%). In contrast, Tg2576 mice pre-treatment and vehicle-treated showed poor performance on the test with a statistically significant lower percentage alternation of 52.82±4.7% and 40.4±5.3% respectively. The DC-101-treated Tg2576 mice were significantly different compared to vehicle-treated Tg2576 mice and DC101-treated WT however DC101-treated Tg2576 mice were indistinguishable from pre-treatment Tg2576 mice (FIG. 15b).

In contextual fear conditioning for the associative memory assessment, mice were seen to “freeze” (remain stationary) after being placed in an environment where they had previously received an electric shock. Pre-treatment, vehicle-treated and DC-101 treated WT (B6/SJL) animals exhibited good associative memory with freezing percentages of 15.96±3.2%, 20.5±7.5% and 29.06±12.7% respectively (FIG. 15c). Contrastingly, pre-treatment and vehicle-treated Tg2576 mice showed poor freezing percentages of 3.2±1.6% and 0.98±1.3% respectively. (FIG. 15: Anti-angiogenic drug, DC-101, partially prevents the cognitive decline associated with aged AD mouse model Tg2576). DC-101-treated Tg2576 mice showed a freezing percentage to 9.6±6.2%. This was significantly different from vehicle-treated Tg2576 mice and DC-101-treated WT mice, however, were not significantly different from pre-treatment Tg2576 mice.

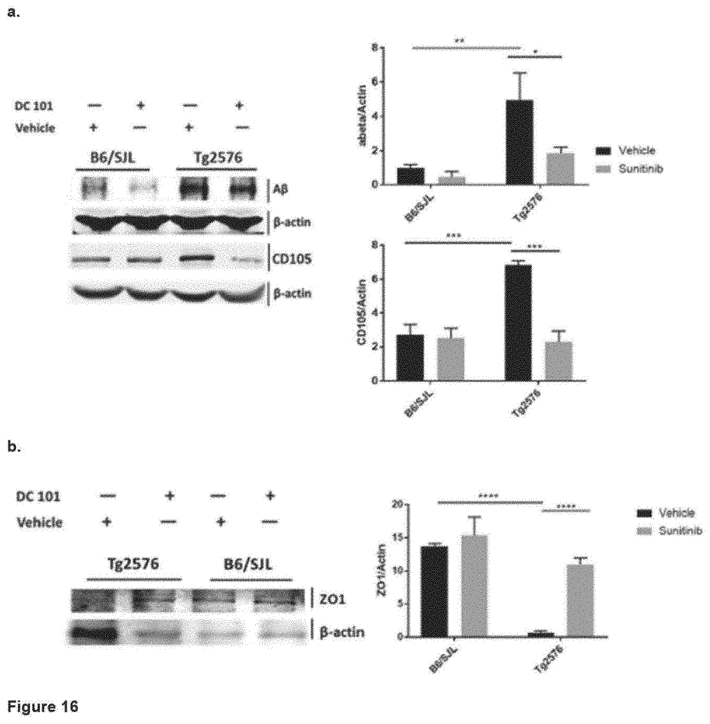

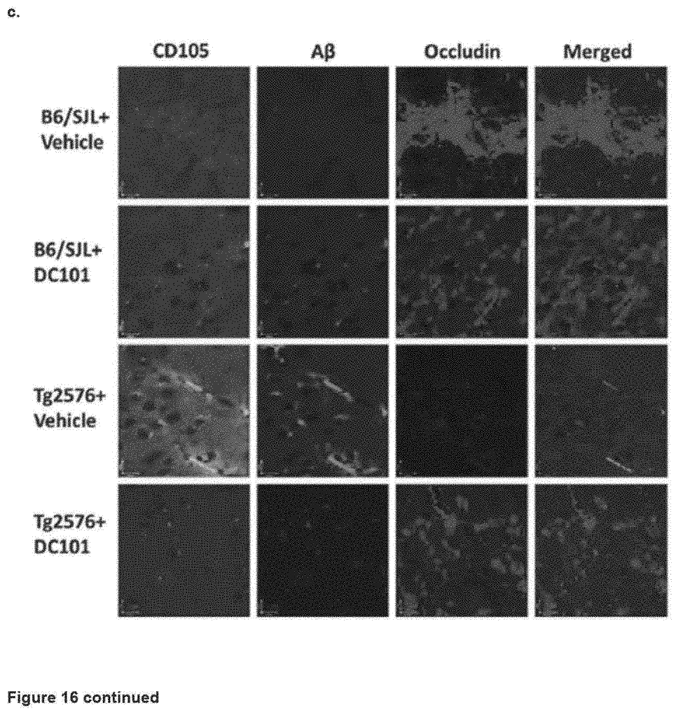

DC-101 reduces expression of cerebral Aβ, angiogenic marker, CD105 and tight junction proteins in aged Tg2576 mice: To assess the effect of the antibody, DC-101, on AD pathology, the brains of treated mice were analysed by semi-quantitative western blotting to look for protein expression of the neoangiogenic marker CD105, as well as of Aβ and the tight junction protein, ZO1. One-month treatment of Tg2576 mice with DC-101 resulted in a decrease in Aβ and CD105 expression levels (FIG. 16a) and increase in the expression in ZO-1 compared to those treated with the vehicle alone (FIG. 16b).

Immunofluorescence analysis of these proteins in both, the cortex and hippocampus of the mouse brains confirmed the western blotting data. FIG. 16c shows representative micrographs of the cortex and hippocampus. Higher amyloid and CD105 staining were observed in the vehicle-treated Tg2576 mice but not in Tg2576 mice that had been treated with DC-101. Reduced occludin expression was observed in the vehicle-treated Tg2576 mice, but after treatment with DC-101 occludin expression was similar to the expression seen in the WT animals.

DC-101 Treatment Reduces the Loss of Tight Junction Structure and BBB Integrity in Aged Tg2576 Mice.

A normal occludin expression pattern, indicated by white arrows in FIG. 17, is strong and continuous. It was observed that the WT mice showed a normal expression pattern of TJPs and thus a low percentage of BBB disruption, irrespective of DC-101 or vehicle treatment. In the vehicle-treated Tg2576 mice, however, there was a disruption of the TJPs that was not seen in Tg2576 mice treated with DC-101.

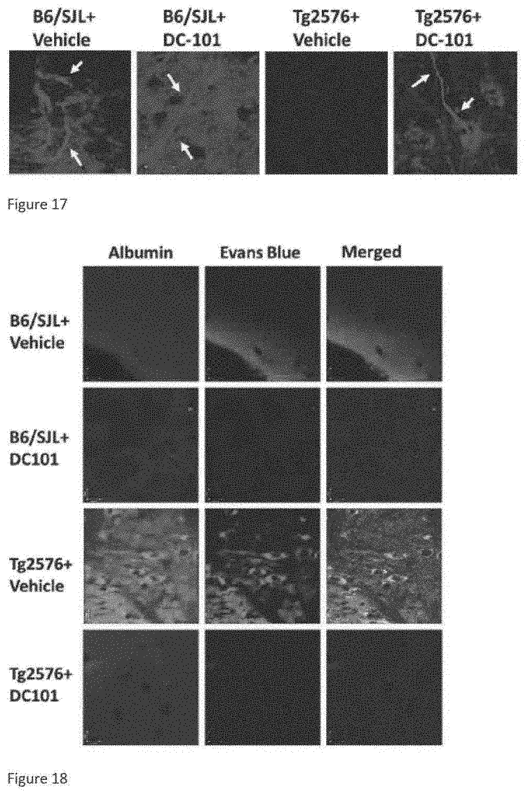

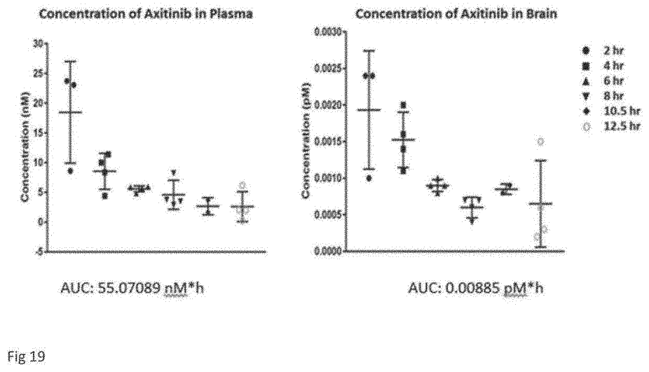

To show that this intact arrangement of the TJP influences the impermeability of the BBB in the DC-101-treated Tg2576 animals, we i.p injected Evans Blue dye in the mice. Evans Blue dye binds to serum albumin, a protein to which the normal BBB is impermeable. A disrupted BBB will allow albumin to move across and enter the central nervous system (CNS), which is indicated by the presence of the dye in the brain. FIG. 18 shows that when Evans Blue dye is injected into mice that have an intact BBB, the dye is unable to cross into and stain the brain. Representative micrographs of the cortical and hippocampal regions of the brain in FIG. 18 show minimal amounts of albumin in the WT brains, indicating an intact BBB. In contrast, substantial presence of both Evans blue and albumin is apparent in the brains of vehicle-treated Tg2576 mice, implying a disrupted and permeable barrier. The brains of the DC-101-treated Tg2576 mice appear similar to those of the WT mice, indicating a functional BBB.

### DISCUSSION

AD mouse model Tg2576 mice pre-treatment at 10 months of age showed a significant cognitive decline compared to age-matched WT littermates, particularly in the case of spatial awareness, associative memory, working memory and reference memory. A similar result was observed when compared 11-months-old vehicle-treated Tg2576 mice to vehicle-treated WT mice after one month of treatment. Modulation of cognition was assessed in aged Tg2576 mice and their age-matched WT littermates after a one-month treatment with either small molecule TKI, Sunitinib or with a VEGFR2 antibody, DC101.

Sunitinib treatment showed no change in the spatial memory, the reference memory or the working memory aspects of cognition in WT mice however an increase in the associative memory was seen in Sunitinib-treated WT mice compared to pre-treatment and vehicle-treated WT mice.

The effect of the drug, after one-month treatment, on Tg2576 mice was intricate. Sunitinib-treated Tg2576 mice showed significantly higher scores of performances compared to the vehicle-treated Tg2576 mice in the various memory tests: open field, Y-maze, fear conditioning and radial arm water maze. However, in certain memory aspects, like associative memory assessed by fear conditioning test or spatial awareness assessed by Y-maze, the Tg2576 mice showed no significant difference compared to the pre-treatment Tg2576 mice. Similarly, DC-101 treatment showed no effect on WT animals when compared to the pre-treatment and vehicle-treated WT animals. In Tg2576 mice DC-101 treatment showed improvement in anxiety levels, locomotion and awareness of a novel environment compared to pre-treatment and vehicle-treated Tg2576 mice however no significant difference was observed between DC101-treated and pre-treatment Tg2576 mice with respect to associative and spatial memory aspects.

The conclusion drawn from these results is that one-month treatment with Sunitinib can improve memory aspects like reference memory, working memory and awareness of a novel environment to promote thigmotaxis and can also prevent the further decline of spatial and associative memory in aged Tg2576 mice. One-month treatment with DC-101 also improves the awareness in a novel environment while preventing further decline of spatial and associative memory in Tg2576 mice.

The molecular and immunohistochemical analysis of AD pathology after one-month of treatment with Sunitinib and DC-101 show that there was a lower expression of Aβ and CD-105 and a higher expression of ZO1 and occludin in vehicle-treated and Sunitinib/DC-101-treated WT mice, as well as Sunitinib/DC-101, treated Tg2576 mice compared to the vehicle-treated Tg2576 mice. It was also observed that the with the one-month Sunitinib and DC-101 treatments there was a higher functionality of the BBB as compared to the vehicle-treated Tg2576 mice. Thus, Sunitinib and DC-101 have the ability to modulate cerebral pathology, cognitive decline and other pathological indications seen in the Tg2576 mouse model of AD and show great potential as therapeutics of AD.

11-month old vehicle-treated Tg2576 mice showed a higher presence of amyloid in the brain compared to their age-matched WT littermates. This was expected as it has been documented in previous studies that in this mouse model Aβ accumulation is initiated between 6-9 months [Kawarabayashi, T., et al., 2001] and plaques are developed by the age of 9 months [Hsiao et al 1996, Lee, K. W., et al., 2009]. Vascular pathology was seen higher in Tg2576 mice compared to WT littermates which include increased microvessel density, as indicated by the higher expression of the neo-angiogenic marker, CD105 and higher BBB permeability as indicated by the higher presence of the Evans blue and immunostained albumin in the CNS. This result was expected as it was shown previously that vascular pathology and BBB disruption were initiated as early as 4 months of age in the Tg2576 mouse model and increase significantly as the mice age compared to age-matched WT littermates (Biron et al 2011). The data presented in this chapter and from other studies suggest that vascular changes are a crucial component of AD pathogenesis.

The data from this chapter demonstrate that vascular pathology is a crucial component of the pathogenesis of AD and its modulation with anti-angiogenic molecules can help to prevent or treat aspects of disease pathology. Since vascular changes were observed in aged Tg2576 animals as opposed to the WT littermates that displayed a normal vasculature, it can be implied that the production of Aβ has a role to play in the initiation of neo-angiogenesis leading to vascular pathology. Secondly, since vascular pathology precedes Aβ accumulation and plaque formation (Biron et al 2011) it can be concluded that vascular pathology directly or indirectly facilitates in this Aβ accumulation and plaque formation. We need to study the vascular aspect of AD pathology in depth to further understand the molecular mechanism and the role that vascular pathology plays in establishing overall AD pathology.

Amyloidgenesis Promotes Dysregulated Neoangiogenesis Leading to BBB Disruption and Other AD Pathologies

An RNA-microarray analysis (data not shown) conducted with samples derived from brain homogenates of 12-month-old vehicle-treated Tg2576 mice and age-matched vehicle-treated WT mice showed global changes in gene expression between the two groups that were related to processes like memory, endothelial proliferation, response to hypoxia and survival. These data provide candidates that could be potential targets to help understand the mechanisms of pathophysiology in AD. To further delve into this, a proteome array was performed on brain protein homogenates from 11-month-old untreated Tg2576 mice and homogenates from age-matched WT mice to look for various effector proteins involved in angiogenesis. In comparison to the WT, the Tg2576 mice showed an overall increase in the pro-angiogenic effectors and a decrease in the anti-angiogenic effectors, with the exception of the anti-angiogenic endostatin, which could be upregulated as a compensatory mechanism for the overall increase in angiogenesis.

The disclosure of all patents, publications, including published patent applications, and database entries referenced in this specification are expressly incorporated by reference in their entirety to the same extent as if each such individual patent, publication, and database entry were expressly and individually indicated to be incorporated by reference.

### REFERENCES

Abbott, A., and Dolgin, E. (2016). Nature 540, 15-16.
BC_Cancer_Agency (2015). Drug Index (Professional): The BC Cancer Agency's Cancer Drug Manual.
Bell, R. D et al. (2010). Neuron 68, 409-427.
Biron, K. E., et al. (2013). Sci Rep 3, 1354.
Biron, K. E., et al. (2011). PLoS One 6, e23789.
Chaahat Singh, C. G. P. a. W. A. J. (2017). Physiologic and Pathologic Angiogenesis, D.
Simionescu, ed. (IntechOpen), pp. 93-109.
Chen, P. C., et al. (2010). J Clin Invest 120, 4353-4365.
Claassen, J. A., and Zhang, R. (2011). J Cereb Blood Flow Metab 31, 1572-1577.
Cummings, J. L., et al. (2014). Alzheimers Res Ther 6, 37.
Desai, B. S., et al. (2009). J Neural Transm (Vienna) 116, 587-597.
Dickstein, D. L., et al. (2006). FASEB J 20, 426-433.
Dobson, K. R., et al. (1999). Calcified Tissue International 65, 411-413.
Gama Sosa, M. A., et al. (2010). The American Journal of Pathology 176, 353-368.
Hansen, T. M., et al. (2010). Cell Signal 22, 527-532.
Hsiao, K., et al. (1996). Science 274, 99-102.
Hsiao, K. K., et al. (1995). Neuron 15, 1203-1218.
Iadecola, C. (2004). Nat Rev Neurosci 5, 347-360.
Jefferies, W. A., et al. (2013). Alzheimers Res Ther 5, 64.
Kawarabayashi, T., et al. (2001). The Journal of Neuroscience 21, 372-381.
Kook, S. Y., et al. (2013). Tissue Barriers 1, e23993.
Matsumoto, T., et al. (2005). EMBO J 24, 2342-2353.
O'Hara, S. D., and Garcea, R. L. (2016). MBio 7.
Papetti, M., and Herman, I. M. (2002). Am J Physiol Cell Physiol 282, C947-970.
Pfeifer, M., et al. (2002). Science 298, 1379.
Ruitenberg, A., et al. (2005). Ann Neurol 57, 789-794.
Sakurai, Y., et al. (2005). Proc Natl Acad Sci USA 102, 1076-1081.
Shalaby, F., et al. (1995). Nature 376, 62-66.
Stewart, S., et al. (2011). Journal Of Alzheimer's Disease: JAD 26, 105-126.
Teichert, M., et al. (2017). Nature communications 8, 16106-16106.
Thurston, G., and Daly, C. (2012). Cold Spring Harb Perspect Med 2, a006550.
Ujiie, M., et al. (2003). Microcirculation 10, 463-470.
Vagnucci, A. H., Jr., and Li, W. W. (2003). Lancet 361, 605-608.
Westerman, M. A., et al. (2002). The Journal of Neuroscience 22, 1858-1867.
WHO (2015). Dementia factsheet http://wwwwhoint/mediacentre/factsheets/fs362/en/.
Wimo, A., et al. (2017). Alzheimers Dement 13, 1-7.
Yuan, H. T., et al. (2009). Mol Cell Biol 29, 2011-2022.
Zlokovic, B. V. (2008). Neuron 57, 178-201.
Zlokovic, B. V. (2011). Nat Rev Neurosci 12, 723-738.
Lampron, A., et al. Brain, Behavior, and Immunity, 2011. 25: p. S71-S79.
Magga, J., et al., J of Cellular and Molecular Medicine, 2012. 16(5): p. 1060-1073.
Lee, K. W., et al., Journal of Neurochemistry, 2009. 108(1): p. 165-175.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A method of treating and/or delaying the onset of Alzheimer's disease by inhibiting Angiopoietin-2 mediated Tie-2 angiogenic pathway comprising administering to the subject an effective amount of an inhibitor of the Angiopoietin-2 mediated Tie-2 angiogenic pathway.

2. A method of treating and/or preventing cognitive decline by inhibiting cerebral neo-angiogenesis, comprising administering to the subject an effective amount of an inhibitor of the Angiopoietin-2 mediated Tie-2 angiogenic pathway, wherein cerebral neo-angiogenesis is inhibited by inhibiting the Angiopoietin-2 mediated Tie-2 angiogenic pathway.

3. The method of claim 2, wherein the cognitive decline relates to one or more of the following: spatial awareness, exploration, associative memory, working memory and reference memory.

4. The method of claim 1, wherein the inhibitor of the Angiopoietin-2 mediated Tie-2 angiogenic pathway is a natural product or extract.

5. The method of claim 4, wherein the product or extract is selected from the group consisting of extracts from Acacia aulacocarpa, Artemisia annua (Chinese wormwood), Viscum album (European mistletoe), Curcuma longa (curcumin), Scutellaria baicalensis (Chinese skullcap), resveratrol and proanthocyanidin (grape seed extract), Magnolia officinalis (Chinese magnolia tree), Camellia sinensis (green tea), Ginkgo biloba, quercetin, Poria cocos, Zingiber officinalis (ginger), Panax ginseng, Rabdosia rubescens hora (Rabdosia), Chinese destagnation herbs, Forsythia suspensa, Forsythia fructus and Voacanga africana.

6. The method of claim 1, wherein the inhibitor of the Angiopoietin-2 mediated Tie-2 angiogenic pathway is an antibody that binds ANG-2 and/or TIE2 and fragments and variants thereof; non-antibody peptide that binds ANG-2, a non-antibody peptide that binds TIE2 and a small molecule inhibitor of ANG-2 or TIE2.

7. The method of claim 1, wherein the modulator of the Angiopoietin-2 mediated Tie-2 angiogenic pathway is Trebananib, Vanucizumab, MEDI3617, Nesvacumab, Rebastinib, MGCD-265; Pexmetinib; CEP-11981, BAY-826, 3,21-dioxo-olean-18-en-oic acid, Altiratinib, 4-[4-(6-methoxy-2-naphthalenyl)-2-[4-(methylsulfinyl)phenyl]-1H-imidazol-5-yl]-pyridine, AB536, 2×CON and L1-7.

8. The method of claim 1, further comprising administration of additional therapeutic agents.

9. A diagnostic method for identifying subjects at risk of developing Alzheimer's disease or having early stage Alzheimer's disease or other neurological diseases, comprising screening for biomarkers from the Angiopoietin-2 mediated Tie-2 angiogenic pathway.

10. The method of claim 2, further comprising treating and/or preventing cognitive decline by inhibiting cerebral neo-angiogenesis comprising administering an inhibitor of VEGFR selected from the group consisting of Axitinib, Sunitinib and DC101 to a subject.

11. The method of claim 10, wherein the cognitive decline relates to one or more of the following: spatial awareness, exploration, associative memory, working memory, reference memory, anxiety, depression, addictive behaviour, obsessive behaviour, compulsive behaviour and repetitive behaviour.

12. The method of claim 1, wherein said subject is a human or animal.


SEQUENCE LISTING (3 sequences)

SEQ ID NO: 1 - DNA, 29 bp, Artificial Sequence
  Primer 1502
gtggataacccctcccccagcctagacca

SEQ ID NO: 2 - DNA, 25 bp, Artificial Sequence
  Primer 1503
ctgaccactcgaccaggttctgggt

SEQ ID NO: 3 - DNA, 28 bp, Artificial Sequence
  Primer 1501
aagcggccaaagcctggagggtggaaca